SEQUENCE LISTING (56 sequences)

SEQ ID NO: 1 - DNA, 28 bp, Artificial
  primer 1, target polynucleotide
gcaactccaggcactccatgagtcatgg

SEQ ID NO: 2 - DNA, 28 bp, Artificial
  primer 2, target polynucleotide
gcaactccaggtggcgactcatacgtca

SEQ ID NO: 3 - DNA, 28 bp, Artificial
  primer 3, target polynucleotide
gcaactccagctcagggggactctatca

SEQ ID NO: 4 - DNA, 28 bp, Artificial
  primer 4, target polynucleotide
gcaactccaggctctagggcaagtctca

SEQ ID NO: 5 - DNA, 28 bp, Artificial
  primer 5, target polynucleotide
gcaactccaggcagactctggatctcag

SEQ ID NO: 6 - DNA, 28 bp, Artificial
  primer 6, decoy polynucleotide
gcaactccaggcagcatgaggtctcatc

SEQ ID NO: 7 - DNA, 28 bp, Artificial
  primer 7, decoy polynucleotide
gcaactccaggcagcaggagtctcattc

SEQ ID NO: 8 - DNA, 28 bp, Artificial
  primer 8, decoy polynucleotide
gcaactccagggtggctcagcaatactc

SEQ ID NO: 9 - DNA, 28 bp, Artificial
  primer 9, decoy polynucleotide
gcaactccagctcagggcagtgatctca

SEQ ID NO: 10 - DNA, 28 bp, Artificial
  primer 10, decoy polynucleotide
gcaactccaggggggcacacattctatc

SEQ ID NO: 11 - DNA, 15 bp, Artificial
  Constant sequence
ctgtaagcggatgcc

SEQ ID NO: 12 - DNA, 20 bp, Artificial
  Coding sequence
cctcgcgcgtttcggtgatg

SEQ ID NO: 13 - DNA, 15 bp, Artificial
  Universal sense primer
ggcatccgcttacag

SEQ ID NO: 14 - DNA, 38 bp, Artificial
  Probe anti-1
tggatcccgcacacgactgaccatgactcatggagtgc

SEQ ID NO: 15 - DNA, 38 bp, Artificial
  Probe anti-2
tggatcccgcacacgactgatgacgtatgagtcgccac

SEQ ID NO: 16 - DNA, 38 bp, Artificial
  Probe anti-3
tggatcccgcacacgactgatgatagagtccccctgag

SEQ ID NO: 17 - DNA, 38 bp, Artificial
  Probe anti-4
tggatcccgcacacgactgatgagacttgccctagagc

SEQ ID NO: 18 - DNA, 38 bp, Artificial
  Probe anti-5
tggatcccgcacacgactgactgagatccagagtctgc

SEQ ID NO: 19 - DNA, 38 bp, Artificial
  Probe anti-6
tggatcccgcacacgactgagatgagacctcatgctgc

SEQ ID NO: 20 - DNA, 38 bp, Artificial
  Probe anti-7
tggatcccgcacacgactgagaatgagactcctgctgc

SEQ ID NO: 21 - DNA, 38 bp, Artificial
  Probe anti-8
tggatcccgcacacgactgagagtattgctgagccacc

SEQ ID NO: 22 - DNA, 38 bp, Artificial
  Probe anti-9
tggatcccgcacacgactgatgagatcactgccctgag

SEQ ID NO: 23 - DNA, 38 bp, Artificial
  Probe anti-10
tggatcccgcacacgactgagatagaatgtgtgccccc

SEQ ID NO: 24 - DNA, 10 bp, Artificial
  Inverse complementary oligonucleotides of the 5′universal region of the markers
ctggagttgc

SEQ ID NO: 25 - DNA, 20 bp, Artificial
  primer 1, marker
agtcgagagccgattccgct

SEQ ID NO: 26 - DNA, 20 bp, Artificial
  primer 2, marker
gtccgagcaaaggcttccgt

SEQ ID NO: 27 - DNA, 20 bp, Artificial
  primer 3, marker
agacccgtgggctccattag

SEQ ID NO: 28 - DNA, 20 bp, Artificial
  primer 4, marker
ccacccagagggcttaggtt

SEQ ID NO: 29 - DNA, 20 bp, Artificial
  primer 5, marker
atcccacgagggtgatctcg

SEQ ID NO: 30 - DNA, 20 bp, Artificial
  primer 6, marker
ggaatccgaccgtgcatgtc

SEQ ID NO: 31 - DNA, 20 bp, Artificial
  primer 7, marker
cagagacgtgacccgctgtt

SEQ ID NO: 32 - DNA, 20 bp, Artificial
  primer 8, marker
gacccaggggtacattctcg

SEQ ID NO: 33 - DNA, 20 bp, Artificial
  primer 9, marker
aaacgagcccgttccgtgtg

SEQ ID NO: 34 - DNA, 20 bp, Artificial
  primer 10, marker
gggagccccagcattatcgt

SEQ ID NO: 35 - DNA, 20 bp, Artificial
  primer 11, marker
ggacgtgaacgcatccgtct

SEQ ID NO: 36 - DNA, 20 bp, Artificial
  primer 12, marker
ggctgaaggccactactctg

SEQ ID NO: 37 - DNA, 20 bp, Artificial
  primer 13, marker
gtaggtagcacaccgtcgct

SEQ ID NO: 38 - DNA, 20 bp, Artificial
  primer 14, marker
cagccaggagatgtccgtct

SEQ ID NO: 39 - DNA, 20 bp, Artificial
  primer 15, marker
gtccccaggtgagatcatcg

SEQ ID NO: 40 - DNA, 20 bp, Artificial
  primer 16, marker
cgagggaccagcttccgtat

SEQ ID NO: 41 - DNA, 20 bp, Artificial
  primer 17, marker
gccagtcgcaggcatgattc

SEQ ID NO: 42 - DNA, 20 bp, Artificial
  primer 18, marker
cgccagggtctcagtcgtaa

SEQ ID NO: 43 - DNA, 20 bp, Artificial
  primer 19, marker
gagcatagccgacgtcttcg

SEQ ID NO: 44 - DNA, 20 bp, Artificial
  primer 20, marker
gtagagtgacacgtcgctcc

SEQ ID NO: 45 - DNA, 960 bp, Artificial
  coding marker
cagaagaatgcacgctctttaacgcttcgccctaaaatgggccatgactattgagaatacgataccttcccgcgttagcatcccttccctgatgctggtagatctacaccatctgtacgggagataaggctggctgtgcgcttagacgggaacttggaccggaagaatgcgtacagccttacgcgcatccgagtcgtcacctaccacacgctcatgcgcactttacgggtaaaaagtgttaatcgtaacagtgtcgggaccactcctatgctaataccagcgtggtccagtgacgtttttacatagtaggtgctctaatcttgcaaaccaccgtttcattatctgttattctcccttgctaatggcccgctcagcaccgggtgttcccagaggagagctcccagccacgttgcagcgagcgggctgtcgaaagtataaagttcttacacattctatgcgtatcatttctcctacggatctggagctaatccggtacgcagcttacgcacaatggcataagctgtgacgtgggatagatagtactctctacaaacgtacagagcagtgttatcgataccccccgcagccaattactcataaatccgacacagcaacgcccattttcagttttcggtatacgtgcgggctcctcatatgtattaacgttgagtgactctgcagtccgattggatgttggttccatccgctatcgtagagtcctatcgaaatccacaaatctgtgcgatttgacgtacatttcgtatcggcggtagttacgcaagctagcactaccatagtagtatcgttattcgggcttgacaacctcgaaggcgtggggaagaagcgtcagtatcttcgcaagaaatgaggaagaggtaccaataagcgaatgggcccgaaactacgtcctcgcagagggcgatcgatcggcgattagagtctggggggtagttcagcaca

SEQ ID NO: 46 - DNA, 20 bp, Artificial
  Sequence of the Mark (Corresponding target markers primer 4, 6, 8, 10, 12)
cgaaataatctgcccggtct

SEQ ID NO: 47 - DNA, 20 bp, Artificial
  Sequence of the Mark (Corresponding target markers primer 2, 6, 10, 14, 17)
actcgtttagggaagctcta

SEQ ID NO: 48 - DNA, 20 bp, Artificial
  Sequence of the Mark (Corresponding target markers primer 1, 5, 9, 13, 16)
agcgcatgatatatagtacc

SEQ ID NO: 49 - DNA, 20 bp, Artificial
  Sequence of the Mark (Corresponding target markers primers 3, 12, 13, 14, 15)
ggtactaagagtggcattgc

SEQ ID NO: 50 - DNA, 20 bp, Artificial
  Sequence of the Mark 1 (table 3.3)
atttggaggcccgaatacaa

SEQ ID NO: 51 - DNA, 20 bp, Artificial
  Sequence of the Mark 2 (table 3.3)
agccccataagacgcgctaa

SEQ ID NO: 52 - DNA, 20 bp, Artificial
  Sequence of the Mark (Corresponding target markers Primers 4, 6, 8, 10, 12)
cgaaataatctgcccggtct

SEQ ID NO: 53 - DNA, 20 bp, Artificial
  Sequence of the Mark (Corresponding target markers primers 2, 6, 10, 14, 17)
actcgtttagggaagctcta

SEQ ID NO: 54 - DNA, 20 bp, Artificial
  Sequence of the Mark (Corresponding target markers Primers 1, 5, 9, 13, 16)
agcgcatgatatatagtacc

SEQ ID NO: 55 - DNA, 20 bp, Artificial
  Sequence of the Mark (Corresponding target markers primers 3, 12, 13, 14, 15)
ggtactaagagtggcattgc

SEQ ID NO: 56 - DNA, 15 bp, Artificial
  Universal sense primer (example 3)
acgttgcagcgagcg

(19) United States
(12) Patent Application Publication
(10) Pub. No.: US 2011/0207125 A1
(43) Pub. Date: Aug. 25, 2011

(54) METHOD FOR LABELLING A PRODUCT USING A PLURALITY OF POLYNUCLEOTIDES, METHOD FOR IDENTIFYING THE LABELLING AND LABELLED PRODUCT

(75) Inventors: Alexandre Jacob, Le Mee Sur Seine (FR); Carlosse Keumeugni Kwemo, Bois Colombes (FR); Sylvain Loric, Brunoy (FR); Stephane Moutereaux, Marolles en Brie (FR); Nicolas Delacotte, L'Hay -les- Roses (FR)

(73) Assignees: BIOQUANTA, Paris (FR); BIOQUANTA CORP., Aurora, CO (US)

(21) Appl. No.: 12/937,754

(22) PCT Filed: Apr. 10, 2009
(86) PCT No.: PCT/FR2009/000422

§ 371 Date: Apr. 5, 2011

(30) Foreign Application Priority Data
Apr. 14, 2008 (FR) 0802044

(51) Int. Cl. C12Q 1/68; C07H 1/00; C07H 21/00; G01N 33/53

(52) U.S. Cl. 435/6.11; 536/25.32; 536/23.1; 436/94

(57) ABSTRACT

A method for labeling a product includes a step of adding on or in the product a plurality of single-stranded polynucleotides, which plurality of polynucleotides includes at least one target polynucleotide constituted of a single-stranded polynucleotide of predetermined length and sequence, and decoy polynucleotides which have identical or different predetermined lengths and identical or different predetermined sequence, which decoy polynucleotides have a length or lengths identical to or different from and sequences different from the sequence of the at least one target polynucleotide, wherein each of the target and decoy polynucleotides does not hybridize with any of the other polynucleotides of the plurality of polynucleotides and wherein the polynucleotides of the plurality of polynucleotides are deoxyribonucleic or ribonucleic acid sequences, respectively having the same proportion of the four, natural or modified, bases A, C, G, and T, or A, C, G and U.

### TECHNICAL FIELD

The present invention relates to a method for labeling a product, to a method for identifying the labeling and to a product labeled through the method of the invention. The labeling used in the present invention is based on single-stranded nucleic acids.

The present invention makes it possible to distinguish an authentic product from a counterfeit product. The invention makes it is particularly possible to label the authentic product so as to be able to track it and identify it.

Counterfeiting, or the illegal reproduction of objects, particularly high-added value products, or high-end products, leads to serious financial consequences for companies, but also in terms of employment, food security and even social life. That is why manufacturers are striving to combat this bane, by developing new labeling and authentication techniques for their products in order to track down and destroy counterfeit products.

The references between square brackets ([x]) refer to the list of references at the end of the examples.

### RELATED ART

One of the methods usually used for detecting and identifying an authentic product is the “bulk” labeling of said product, by incorporating to this product a chemical body or compound that may be identified unilaterally.

This type of labeling must have specific properties: the labeling must be carried out in a transparent manner with respect to the product end user, and should not alter the physico-chemical properties of this product and should not be hazardous to the product end user. It should also be, as far as practice, undetectable and/or impossible to forge so as not to be itself forged at the same time as the product.

Today, there is no reliable technique, stable in time specifically for the very large number of chemical substances that are known to be harsh for markers, such as perfumes, cosmetic creams or on materials such as leather, fabric, etc. Neither is there nowadays labeling techniques that exhibit real deciphering difficulties for counterfeiters.

Thus, there is a real need for a labeling and a labeling method for overcoming these faults, drawbacks and obstacles of the prior art and which make it possible to efficiently combat the counterfeit products, particularly high-end products.

### STATEMENT OF THE INVENTION

The object of the present invention is specifically to propose a labeling solution satisfying this need and resolving the problems of the prior art.

The present invention particularly relates to a method for labeling a product, said method comprising a step of adding on or in said product a plurality of single-stranded polynucleotides, said plurality of polynucleotides comprises:
at least one target polynucleotide comprising a single-stranded polynucleotide of predetermined length and sequence, and
decoy polynucleotides which have identical or different predetermined lengths and identical or different predetermined sequences, said decoy polynucleotides having identical or different length or lengths and sequences different from the sequence of said, at least one, target polynucleotide

wherein each of the target polynucleotide(s) and decoy polynucleotides does not hybridize with any of the other polynucleotides of said plurality of polynucleotides.

In a particular embodiment of the method of the present invention, said plurality of polynucleotides further comprises at least one recognition polynucleotide constituted of a single-stranded polynucleotide of predetermined length and sequence for identifying the nature and sequence of the, at least one, target polynucleotide, wherein each recognition polynucleotide does not hybridize with any of the other polynucleotides of said plurality of polynucleotides.

The following specification is to be considered for the method of the invention such as defined above and for the particular embodiment of the method of the invention.

The labeling of the present invention is thus constituted of said plurality of polynucleotides such as is defined in the present description. These polynucleotides are single-stranded polynucleotides. In the present description, this plurality of polynucleotides is also called “marker”.

The present invention also relates to a labeled product obtainable by the method of the invention and to a method for detecting the labeling of this product.

The present invention particularly relates to the determination of the marker used to implement the method of the invention, the manufacture of these markers, the labeling of products, as well as the techniques for detecting markers in the labeled products. The present invention makes it possible to distinguish a counterfeit product from an authentic product, and even to identify the misappropriations of distribution channels and unauthorized parallel channels.

The polynucleotides of said plurality of polynucleotides may be of several types: they may be ribonucleotide polymers or single-stranded ribonucleic acids (RNA) or deoxyribonucleotides or single-stranded deoxyribonucleic acids (DNA) or a combination of these.

In the present invention, “plurality of polynucleotides” means the set of target, decoy polynucleotide(s), and if need be, recognition polynucleotides. Preferably, according to the present invention, several target polynucleotides, several decoy polynucleotides, and, if need be, several recognition polynucleotides are used. For example without this example being limitative, from 1 to 100 target polynucleotide(s) may be used, for example from 1 to 50, for example from 5 to 50. For example also, without this example being limitative, from 2 to 100 decoy polynucleotides may be used, for example, from 2 to 50. For example, if need be, without this example being limitative, from 1 to 100 recognition polynucleotides may be used, for example from 1 to 50. The choice of the number of polynucleotides depends on the required labeling complexity according to the present invention.

In the present, “target polynucleotide”, means a polynucleotide the sequence of which has been determined and constructed to constitute a reference sequence intended to label the product according to the present invention, then to be searched specifically in this product in order to authenticate it. Preferably, according to the invention, the marker comprises several identical or different target polynucleotides, preferably different. The target polynucleotides are referenced in a confidential targets/products database which establishes the link between the target polynucleotide(s) and the labeled product.

By “confidential database”, is meant a database to which only the labeling manufacturer according to the present invention for a given product and/or only the manufacturer/creator of said product has (have) access (one and/or the other is hereinafter called “the person implementing the present invention”). It is a correspondence base wherein, for each product or product family is assigned a specific signature determined according to the present invention.

In the case where only target and decoy polynucleotide(s) are used for the signature of products, this data or correspondence base may be called “target/products confidential base” or “target/products base”. Thus, if we wish to authenticate a product according to the present invention, a search must be first carried out in the target/products base for the target sequence(s) assigned to said product, and then a search must be carried out to see whether the target sequence(s) is (are) actually present in said product, for example by using one of the methods described below. If the target sequence(s) is (are) actually present, the product is declared authentic. However, if the target sequence(s) is (are) not actually present, the product is declared to be a counterfeit. Of course, this only works if all the authentic products are labeled according to the present invention. The correspondence base may be constructed by the manufacturer of the signature of the present invention and/or by the manufacturer/creator based on a list of signatures according to the present inventions, for example by making each signature correspond to a determined product. In this case, the authentication is direct.

In the present, “decoy polynucleotide” means a polynucleotide the sequence of which has been chosen and constructed to be different from the target sequence(s). The decoy sequences are intended to jumble the labeling according to the invention, but not to be searched in the product in order to authenticate it. Decoy sequences are there to complicate the work of a counterfeiter attempting to reproduce the signature of the present invention. In fact, only the person who implements the present invention knows the target sequence(s). The distinction between one or several specific sequence(s), in this case of the target sequence(s) with a view to reproduce them, among a mixture of target and decoy sequence(s), with a view to reproduce them, is made even more difficult if not impossible that, according to the invention, the decoy sequences are present, that target(s) and decoys are short sequences, single-stranded and do not hybridize with each other. The more the decoys, the more difficult the reproduction of the signature in its entirety is, and statistically, there is less chance to randomly determine the sequence(s) of the target polynucleotide(s). The sequences of these decoy polynucleotides are also known by the person implementing the present invention, however, these polynucleotides are not in a database to be assigned to a product.

In the present, “recognition polynucleotide” means a polynucleotide of predetermined sequence and length which enables the identification of the target polynucleotide(s). The latter may have a nature or be present in concentrations that facilitate a rapid search and identification, serving as first authentication test: their absence is a first sign of a counterfeit. It is one or several polynucleotide(s) the sequence(s) of which has (have) been chosen and constructed to constitute a code used in a confidential database for identifying the target polynucleotide(s) or “target identification base” intended to give information about the number, the nature, the sequence and length of the target polynucleotide(s). When there are several recognition polynucleotides, it is possible to speak of “a set of recognition polynucleotides”. In this case, in the target identification base for each recognition polynucleotide or set of recognition polynucleotides, is assigned a target polynucleotide or a set of target polynucleotides determined according to the present invention. The sequences of recognition polynucleotides are only known by the person implementing the present invention. The presence of recognition polynucleotide(s) in the signature of the present invention is optional; it corresponds to a particular embodiment of the method of the invention. If one or several recognition polynucleotide(s) is (are) used in the signature according to the invention, of a product, a confidential base for identifying the targets is created by the person implementing the present invention. This base makes it possible to identify the target polynucleotide(s) present in a product to be authenticated based on the recognition polynucleotide(s). In this case, the authentication of a product is indirect. In fact, if we wish to authenticate a product supposed to have a signature according to the invention, we identify the recognition polynucleotide(s) according to one of the methods of the present invention described below, then search in the target identification database the target sequence(s) assigned to said product, then search whether the target sequence(s) is (are) actually present in said product, for example by using one of the methods described below. If the target sequence(s) is (are) actually present, the product is declared authentic. However, if the target sequence(s) is (are) not present, the product is declared to be a counterfeit. It is to be noted that in this embodiment of the present invention there are two steps of identifying polynucleotides before authenticating a product, thus making the task of possible counterfeiters more complicated. Of course, this only works if the authentic products are labeled according to the present invention. According to the invention, the sequence of recognition polynucleotides may contain a sub-sequence carrying the code that enables the user of the invention to find which are the target polynucleotides and which are the decoy polynucleotides based on a target recognition base. Thus, during the authentication process of a product, these recognition polynucleotides are extracted from the product to be authenticated, or directly identified in or on the product. Once the nature of the recognition polynucleotides detected—for example, without this example being limitative, the identification of the target polynucleotides may be achieved by the reading of the sequence of a recognition polynucleotide, or by the identification of an array of recognition polynucleotides present among a plurality of putative encoding polynucleotides—the user of the present invention will refer to a correspondence table (target polynucleotides recognition base), and read therein the nature of the target polynucleotides theoretically present in the product to be authenticated. This table may for example, and without this example being limitative, be stored in a secured and computerized database (enabling to ensure confidentiality), which database has been created during the labeling of the product, and in which appear the pairs (“code of recognition polynucleotides read”—“target polynucleotides to search for”). Thus, after having referred back to this table, the user of the invention may deduce the exact nature of the target markers which should be present in the product to authenticate. The target polynucleotides are thus, extracted, then identified. It the detected target polynucleotides correspond exactly to the theoretical code read in the table, then the product is authenticated. If other target polynucleotides are present, the user of the invention may suspect a mix of labeled products. If the detected target polynucleotides are entirely different from those expected, it may be a counterfeit. If there are no recognition polynucleotides used in a signature according to the invention, only a targets/products database is useful.

In the present invention, the polynucleotides of the plurality of polynucleotides may be designed for example by methods known by the skilled person, for example with a software implementing an algorithm such as presented hereafter, such that, for a given polynucleotide, selected among the plurality of polynucleotides constituting the labeling, no other polynucleotide of this plurality, nor any reversed complementary polynucleotide of these polynucleotides is constituted by a sequence of polynucleotides complementary to this given polynucleotide. Thus, no double-stranded complex, for example a nucleic acid duplex, as for example a double helix of DNA, nor any hybridization between the polynucleotides of the signature of the present invention may be formed including at the temperature and in the molecular conditioning environment of the product, and at the temperature and in the polynucleotide molecular development environment.

By “formation of a double-stranded complex”, is meant a pairing of thermodynamically stable complementary nucleotides, including in the aforementioned conditions.

By “reversed complementary polynucleotide” of a given polynucleotide, is meant a new polynucleotide, existent or theoretic, wherein each nucleotide of the given polynucleotide is replaced by a complementary nucleotide being able to pair with the first, as for example an adenine replacing a thymine, or a thymine replacing an adenine, or a cytosine replacing a guanine, or a guanine replacing a cytosine in the case of a deoxyribonucleic acid polynucleotide.

By “hybridization” is meant the association by non covalent linkage of two complementary simple-stranded polynucleotides. This hybridization may be perfect, that is to say that the sequences are entirely complementary, or imperfect, that is to say that the sequences are not entirely complementary but sufficiently complementary to hybridize with each other and form a double-stranded structure.

In the present invention, by “non-hybridization” is meant the non association by non covalent linkages of two single-stranded polynucleotides because they are not complementary and/or because the complementarity is not sufficient for the formation of a double strand.

It is worth noting that the plurality of polynucleotides of the marker of the present invention to be incorporated in a product or a substance of interest are not all assigned to a database for directly or indirectly authenticating a product. Thus, it is possible to only use a restricted number of target polynucleotides, these target polynucleotides being those that are sought during the authentication of a product, and incorporate them in the product at the same time as a large number of decoys, and if need be, of recognition polynucleotides, whereof the sequences do not correspond to those of the target polynucleotides. Thus, the target polynucleotide(s) may be “drowned” in a mass of decoy polynucleotides, extraordinarily confusing the issue in the case of an ill-intentioned attempt at decoding target polynucleotides with a view to reproducing the signature. Furthermore, according to the particular embodiment of the present invention, wherein recognition polynucleotides are used, only the reading and decrypting of the code carried by the recognition polynucleotides can incriminate, among a combination of target polynucleotides and decoy polynucleotides, which actually correspond to the target sequences, and by elimination which are only decoys intended to mislead the counterfeiter.

The labeling method of the present invention may be achieved by means of markers which are desoxy and/or ribonucleic acids. Said polynucleotides of the plurality of polynucleotides may hence be single-stranded deoxyribonucleic acid sequences or single-stranded ribonucleic acid sequences or a combination of deoxyribonucleic acid and acid sequences. A marker in accordance with the present invention may hence be composed of current bases, called “natural bases” for example those present in DNA: adenine, guanine, thymine, cytosine, or in the RNA: adenine, guanine, uracil, cytosine (see for example Molecular Cloning, Maniatis, Cold Spring-Harbor, 2^{nd} edition, pp C3 to C14 [1]). A marker in accordance with the present invention may also comprise less frequent natural or synthetic compounds, called “modified bases”, as for example the dihydrouridine (DHU), inosine, or pseudo uracil which result from modifications, for example a deamination, carried out on the previously presented bases. Nitrogenous bases may be constituted from natural isotopes and/or stable isotopes of different atomic masses and/or be modified in order to establish a number of hydrogen linkages different from normal during hybridization processes.

According to a particular embodiment of the invention, the sequences of deoxyribonucleic acid may comprise, in their sequence, the same proportion of the four natural or modified bases A, C, G and T. According to another particular embodiment of the present invention, the sequences of ribonucleic acids may contain, in their sequence, the same proportion of the four, natural or modified, bases A, C, G and U. According to yet another embodiment of the invention, including or not the two previous embodiments, the set of polynucleotides composing the labeling have the same number of nucleotides, and the same molecular weight. This particular embodiment of the invention enables advantageously to make even more difficult if not impossible for a possible counterfeiter to separate and identify the polymers. For example, the separation and identification according to molecular size and/or weight thanks to techniques such as electrophoresis, for example on agarose gel or polyacrylamide and/or mass spectrometry is impossible to carry out on a signature according to the latter embodiments, especially the last one.

For example, in the labeling of the present invention, a single-stranded polynucleotide (or oligomer) of 20 nucleotides, each of the nucleotides being chosen among 4 possible bases enables to carry out 420 different sequences, namely, around 1.1×1012 combinations, that is to one trillion combinations. The probability for extracting a target marker, according to the invention, randomly for example, a plurality of polynucleotides of size 20 in a labeling in accordance with the present invention, and that this marker is that which has been assigned to the product in the targets/product database is thus practically null.

Furthermore, the labeling of the present invention is composed of several target molecules of predetermined length and sequence, of several decoys, and if need be of several recognition polynucleotides, which at the same time ensure a very high security and a remarkable inviolability of the labeling.

By their nature, the polynucleotides used may thus comprise for example an oriented combination of 4 nitrogenous bases whereof the nature is to be defined by the person implementing the present invention. This combination, which is at the origin of the specificity of each polynucleotide of the marker of the present invention, and which can carry the information relative to the labeled product, may be calculated in a computerized manner, according to the needs (code complexity, information type which the markers carry) as well as the physic-chemical properties that these markers exhibit (hybridization properties, molecular mass, size of the fragments, composition in nitrogenous bases).

According to the product to be labeled and the detection technique of the considered labeling, one or several target polynucleotide(s) may be used. The invention hence allows for a considerable number of signature or labeling variants/alternatives. May be cited, by way of non limitative example, the following different forms for the target nucleotides of the first set:
one or several single-stranded target polynucleotide(s) of large size(s), that is to say comprising for example from 500 to 5000 nucleotides or bases;
one or several single-stranded target polynucleotide(s) of small size(s), that is to say comprising for example from 5 to 200 nucleotides or bases, for example from 5 to 50;
one or several single-stranded target polynucleotide(s) of average size(s), that is to say comprising for example from 201 to 499 nucleotides or bases;
one or several single-stranded target polynucleotide(s) having a constant end sequence and a variable end sequence,
one or several single-stranded target polynucleotide(s) inserted (or determined) in polynucleotides of larger sizes, these inserted target polynucleotides hence forming a larger part of polynucleotides,
one or several single-stranded circular polynucleotide(s), or
a combination of these different forms.

According to the invention, at least two target polynucleotides may be used, one being a circular polynucleotide and the other a linear polynucleotide. A plurality of target circular or linear polynucleotides or a mixture of these may be used, according to the chosen labeling complexity by the person implementing the present invention.

According to a particular embodiment of the present invention, when some or a set of single-stranded polynucleotides are linear, they may comprise a variable end from one polynucleotide to another and a constant end from one polynucleotide to another. By “constant end” is meant a part of the polynucleotide sequence including one of the two ends of said sequence and exhibiting a predetermined and constant sequence, that is to say identical for a part of the target sequences or for all the target sequences of the marker of the present invention. By “variable end” is meant a part of the polynucleotide sequence including the other of the two ends of said sequence and exhibiting a predetermined sequence variable from one target sequence to another in the marker of the present invention. This presents a specific advantage for the target polynucleotides. In fact, in order to detect or decrypt the labeling for the purpose of identifying an authentic product, as described here below, one may use a solid support for the decrypting, support whereon polynucleotides complementary to the variable ends of the target polynucleotides are fixed, as for a DNA microarray. The constant ends themselves may be used to highlight the hybridization of target polynucleotides on the solid support, for example by means of biotin/streptavidin. This detection mode is described here below.

The number and nature of target polynucleotides, combined with their size, makes it possible to define the labeling complexity, and in a combinatory manner, the number of possible combinations. The number of possible combinations increases in an exponential manner with the size of these polynucleotides. By choosing a signature composed of one or several polynucleotides among all the same-size polynucleotides, a large number of combinations is conceivable. The probability of reproducing a signature, at random, amongst all those which are possible, is almost null.

According to the invention, the labeling information may consist in:
target polynucleotide sequences, each assigned in a targets/products database to a product, and/or
in one or several combination(s) of target polynucleotide sequences, said combination(s) being assigned in a targets/products database to a product.

Thus, it is possible to use several target polynucleotides of predetermined sequences. It is also possible to choose for example a group or “pool” of 20 polynucleotide sequences of all different and predetermined sequences, and choose, to label a set of given products, a combination for example of 10 sequences among these 20 for each product.

To these target sequences are added decoy polynucleotide sequences in order to constitute the marker in accordance with the present invention. According to the invention, the decoy polynucleotides do not hybridize with target polynucleotides and their role is to make decrypting the labeling of the present invention in order to copy it even more difficult for a counterfeiter. These decoy polynucleotides may be under linear or circular form or a mixture of circular polynucleotides and linear polynucleotides as indicated above for the target polynucleotides. The number of decoy polynucleotides added to the marker depends on the required confusion. Preferably, this number is higher than the number of target polynucleotides. Examples are given above. The decoy polynucleotides are of identical or different length(s) to and from each other, preferably of identical length to the target sequence(s) present in the marker of the present invention, for example, as indicated above for the target polynucleotides, from 15 to 5000 bases, for example from 15 to 200, for example from 20 to 200, for example from 201 to 499, for example from 500 to 5000 bases, or a mixture of these lengths.

To these target and decoy sequences, recognition polynucleotides may be added, enabling, by means of a database, to distinguish between the target polynucleotides from the decoy polynucleotides as indicated above. The number of recognition polynucleotides particularly depends on the complexity of the required labeling. The recognition polynucleotides may be circular or linear. They can be of length(s) identical to or different from one another and of identical length to or of different length from those of the target and decoy polynucleotides, as indicated above for the target polynucleotides from 5 to 5000 bases, for example from 15 to 200, for example from 20 to 200, for example from 201 to 499, for example from 500 to 5000 bases, or a mixture of these lengths.

Thus, in the labeling of the present invention, said polynucleotides of the plurality of polynucleotides may be circular, linear, or a mixture of circular and linear polynucleotides, for example with a free 3′OH end and a free phosphate 5′ end. Preferably, the length of the polynucleotides of the labeling of the present invention is from 5 to 5000 nucleotides, for example from 5 to 100 nucleotides, for example from 5 to 50 nucleotides, for example from 20 to 50 nucleotides.

According to the invention, said polynucleotides of the plurality of polynucleotides are single-stranded polynucleotides. In fact, one of the features of the present invention is that the use of single strands enables to make decrypting the labeling of the present invention more difficult.

The labeling method of the present invention hence enables to manufacture a very important number of markers. Each marker comprises a code constituted of (a) target polynucleotide(s), and if need be, the recognition polynucleotides.

The sequences of polynucleotides of the marker of the present invention may be created empirically, or preferably, particularly for a matter of rapidity, by an appropriate software which can be generated for this purpose. In the last case, it is a computer design or “Design In Silico” of the marker of the present invention.

For determining the sequences of target and decoy polynucleotides, and, if need be of recognition, of the marker of the present invention, use of the following algorithms may be made:
(0) Creation of a set E which contains the set of polynucleotides generated by the labeling.
(1) Random generation of a first polynucleotide p, whereof the size as well as the number of polynucleotides may be defined by the user, p already not belonging to E.
(2) Calculation of the hybridization score according to the algorithm of Smith and Waterman [2] between: the polynucleotide resulting from the concatenation of two polynucleotides p, on the one hand, and each polynucleotide resulting from the concatenation of two polynucleotides chosen from the set of polynucleotides of E as well as their reversed complementary on the other hand.
(3) If the set of scores does not exceed a threshold given by the user, addition of p in E. Return to step (1) as long as E is not of the required size. This threshold is a minimum alignment score above which two sequences are considered as sufficiently identical to hybridize with each other.

Once the sequences of the target and decoy polynucleotides are determined, these target and decoy polynucleotides may be manufactured, by any existing method known by the skilled person. One or several protocol(s) may be used according to the nature of the manufactured polynucleotides and according to the chosen labeling: synthesis of circular and/or linear single-stranded ribonucleic and/or deoxyribonucleic acids, with a variable size for example from 5 bases to 5000 bases. By way of example of usable protocols implementing the present invention, those enabling to synthesize circular single-stranded polynucleotides [3] or insilico synthesis protocols of polynucleotides [4] may be cited.

When sequences of recognition polynucleotides are required for the labeling, their respective sequences may be determined empirically, or by using an algorithm such as described previously, such that the recognition polynucleotides do not hybridize with each other, nor with the target polynucleotides or the decoy polynucleotides.

The labeling of a solution or a compound using ribonucleic or deoxyribonucleic acid markers according to the method of the invention may be achieved in various ways according to the complexity of the required labeling. Each target polynucleotide carries specific information, inherent to its sequence. Each target polynucleotide or combination of target polynucleotide may be used in a unique manner.

The first possible coding level may be that consisting in using one or several target polynucleotides. Several batches of labeled products may thus be tracked by one or several polynucleotide(s) of defined size(s), and predetermined sequence(s) but different from each other.

The second possible coding level is the use of several target polynucleotides, chosen in an initial pool of target polynucleotides. Thus, the code is no longer from each sequence of target polynucleotide but in the combination of target polynucleotides found in a product. Thus, according to the invention, a labeled product may be labeled by n target polynucleotides chosen among a possibility of N different polynucleotides, n being comprised in the interval [0; N].

In all cases, the labeling of the present invention may be completed by a third level of coding, consisting in the use of recognition polynucleotides, thus indicating, by means of a target recognition database, which markers among the target polynucleotides liable to be used by the manufacturer and which may be of different nature and concentration, are present if the product is authentic and labeled with the method of the present invention.

According to the invention, each step of the labeling method of products may be the subject of a specific traceability. This traceability may be ensured by introducing specific information to each step in one or several confidential databases.

Thus, according to the invention, each batch of markers or combination of markers may for example be identified by an alphanumerical identifier. This identifier may appear on the product or by any other optical representation mode: for example bar code, Data Matrix, etc., for example on the container of the batch of markers. This identifier may also serve as an index in a first database where the information of the batch of markers are stored, for example: sequence of each one of the markers composing the batch, respective proportions of the quantities of each marker, manufacturing date, etc.

Each container of batches of markers, tracked by means of its identifiers, may be linked in a database for example to the reference of the order of a client and to the delivery references of this container to said client. A confirmation of receipt from the client may also be entered into this database.

Nucleic acids do not alter anything from the physico-chemical properties of the labeled products. Furthermore, nucleic acids do not have any effect on their container, that is to say the labeled product. Finally, nucleic acids have proved to be very stable in numerous tests carried out by the inventors. This stability property is demonstrated in the examples below.

In a non-exhaustive manner, the single-stranded DNA and RNA markers comprised in the present invention may be included in a very wide panel of products and substances liable to be the object of illegal, abusive reproduction (counterfeiting), illicit trade on a black market and/or of which it is essential to follow the trace (product tracking).

The method of the invention applies to the labeling of all liquid, semi-liquid or solid industrial or consumer products. May be cited, for example, without this list being limitative: fragrances, cosmetics, hygiene products, food products, flavorings, plant extracts, tobacco, beverages, textiles, leathers, medicines, powders, varnishes, inks, paint, chemical products and compounds, and more generally all goods and products liable to be counterfeited.

The invention may apply to an entire range of products from the high-end industry and cosmetics industry: fragrances, eaux de parfums, eaux de toilettes, essential oils, creams, masks, pomades, etc.

In the fields of industrial and consumer products, the invention may also serve to track various substances such as ink, resins, varnishes, paint, dyes, additives, aromas, glues, powders.

In the food-industry field, the present invention can be applied to high-end products which may be a target for counterfeiting or cheating (mixing), such as particularly liquors, spirits, grand crus, or even any product of which it is important to ensure the authenticity for safety reasons for example.

Markers may also be used in the pharmaceutical industry to label and track medicines and other drugs.

The invention may also be used for tracking biological samples in a hospital environment. Such as for example implementing a traceability protocol for blood samples in a biochemical laboratory, tumor samples in an anatomopathology laboratory, or with a view to constitute a human tissue bank directly authenticated which may be kept and tracked for many years in a biological resources centre.

Generally, products can be labeled either for what they stand for, or as a constituent. For example, a paper document may be labeled by means of the ink which has been used on it and which has been previously labeled.

According to the invention, the step of addition of the marker of the present invention may be achieved by any appropriate means enabling to add, to the product that is to be labeled, the polynucleotides constituting the marker of the present invention. The products and substances to be labeled may be labeled in the bulk, by incorporating thereto the markers whereof the final concentration is studied and provided or at the surface of the product. The markers are polymers of ribonucleic or deoxyribonucleic acids having physico-chemical properties deriving from their nature: they are hydrophilic molecules that are charged negatively. According to the nature of the product to be labeled, they may be pre-diluted or directly added to the product. They may also be encapsulated. They even be deposited or incorporated to the surface of the product.

This addition of the marker of the present invention to the product may be achieved by addition of said plurality of polynucleotides in said product during its manufacture and/or in or on (that is to say on the surface) the end product. The present invention thus also relates to a labeled product that may be obtained by the labeling method according to the invention.

According to the invention when the addition of the marker is achieved at the surface of the product to be labeled, it may be carried out for example by dipping the end product in a solution comprising said marker or by spraying or vaporizing such a solution on the end product. Preferably, the solution is a protic solvent such as ethanol, methanol or diethylene glycol, or a polar aprotic solvent such as acetone or tetrahydrofuran. This addition mode is suitable for example for solid products after their manufacture such as fabric, leather, wood, paper, cardboard, tobacco, cigarettes, cigars, etc.

The addition may also be carried out during the manufacture of the product, by mixing said marker with the compounds or components constituting the product. This introduction of the plurality of polynucleotides may be achieved on or in a component of said product. This type of addition is suitable for any product or substance whereof the manufacture goes through a liquid or semi-liquid phase wherein the markers may be incorporated. It can be the case for example for the integral labeling of a cosmetic product or a medicine.

According to a particular embodiment of the present invention, an encapsulation step of said plurality of polynucleotides in lipid vectors may be carried out prior to the step of addition. This encapsulation step enables to maintain the polynucleotides in a favorable environment, or facilitate their future extraction. For example, as encapsulation product, a product selected from the group comprising a cationic lipid vector can be used, such as dioleoxyloxy-propyl-trimethylammonium bromide (DOTMA) and dioleoyl phosphatidyl ethanolamine (DOPE), or polynucleotides complexes with molecules such as polylysine, protamine, or polyethyleneimine (PEI) called polypexes. For example, to that end the technique described in Bioch. Biophys. Acta 1280:1. [5], J. Biol. Chem. 269:2550 [6] or AAPS PharmSci. 2001; 3(3):E21 [7] may be used.

According to another embodiment of the invention, a step of encapsulation and/or protection of said plurality of polynucleotides in lipid vectors or other may be achieved prior to the step of addition. This step further ensures the stability and/or facilitate its recovery.

In the present invention, “protection” means the protection of polynucleotides in the present invention, particularly from any physico-chemical attack from the environment wherein is found the signature of the present invention (fragrance, food) for example in these polymers charged in carbon nanotubes.

Whatever the selected addition mode, the marker of the present invention is preferably added in the product for its labeling in very small concentrations ranging from micromolar to femto-molar. According to the considered detection technique, existent or to come, and the size of the markers, these are added to a variable end concentration, the respective concentrations of each target polynucleotide of the first set able to be different and compose a coding sub-set. According to the invention, the concentration of the plurality of polynucleotides after its addition in said product may be, but without being limited thereto, of 10⁻⁶ moles to 10⁻¹⁸ moles/dm³. This quantity per volume is indicated for a liquid or a solid volume. For a liquid, it corresponds to the quantity of markers mixed with the product by volume unit. For a solid, it corresponds to the quantity of markers mixed with the product by volume unit or deposited at the surface of the product. Brought to the surface of the product, this quantity may also be defined by unite de surface, namely 10⁻⁶ moles to 10⁻¹⁸ moles/dm². These concentrations may be obtained by diluting a more concentrated solution. The solution may be such as defined above.

The present invention also relates to a method for detecting the labeling of a product that could be obtained by the labeling method of the invention, said method comprising an analyzing step of the plurality of polynucleotides enabling to specifically detect the, at least one, target polynucleotide.

Moreover, the invention provides several methods for detecting markers of the present invention. The detection may be achieved outside the laboratory or in it, for example by means of a portable system, for example by means of a DNA microarray especially designed for detecting target polynucleotides of the marker of the present invention.

According to the invention, the analyzing step may be carried out for example by immunodetection. According to the invention, the analyzing step may comprise for example a step of sequencing of the encoding polynucleotides. According to the invention, the analyzing step may for example comprise a retro-transcription of the ribonucleic acid into deoxyribonucleic acid, particularly when the target polynucleotides are ribonucleic acid. According to the invention the analyzing step may comprise a colorimetric, luminescent or fluorimetric detection, coupled with a specific hybridization. In other words, the analyzing step may use a specific means for detecting target polynucleotides.

According to the invention, the analysis technique used may implement the physico-chemical properties, code and specificity of the marker. It may be about for example a sandwich assay technique, a detection technique using DNA microarray, or any other technique known by the skilled person making it possible to detect the presence of polynucleotides and/or to identify them.

In a particular embodiment of the invention, the target and decoy polynucleotides may be small sized polynucleotides, that is to say from 8 to 30 nucleotides/nucleosides and simple stranded. According to this embodiment and advantageously, these polynucleotides given their nature, cannot be used as template strand to be amplified and detected by exponential amplification techniques such as polymerization chain reactions (“PCR”, Polymerase Chain Reaction). Thus, their detection by PCR amplification is impossible. In this embodiment, the detection of polynucleotides may be carried out by hybridization processes and direct detection without amplification and achievable only by the user who knows the signature used. In addition, the counterfeiter who would attempt to extract, amplify and reproduce the plurality of polynucleotides present in the labeled product would be held in check. Moreover and advantageously, the detection methods only comprising steps of hybridization and detection of this hybridization may be implemented much more rapidly a polymerization chain reaction requires several hours, whereas the simple hybridization of polynucleotides is done nearly instantaneously) as far as the detection is specific to the polynucleotides to be detected.

According to the invention, the analysis technique used may be based on the physico-chemical properties, code and specificity of the marker. It may comprise an exponential or linear amplification of the target polynucleotides, any other technique may be used to detect the presence of the markers. In other words, the analyzing step may comprise a linear extension step of the target polynucleotides.

The method of the invention may further comprise the following steps, before the analyzing step:

(a) Sampling the product; and

(b) Extraction of the plurality of polynucleotides of said sample.

Here, it consists in of a means enabling to implement the detection of the labeling of the present invention by sampling the product.

After having been extracted, the presence of markers may be analyzed as indicated above.

According to the invention, the extraction step (b) may be achieved by any technique known by the skilled person to extract the polynucleotides from a sample. The polynucleotides may be extracted according to a protocol depending on the nature of the labeled product. Any type of technique for extracting ribonucleic acid or deoxyribonucleic acid, existent or to come, may be used to extract the polynucleotides from the mass of the labeled product. It may be for example a phenol-chloroform extraction. Extraction techniques usable in the present invention are for example described in Molecular Cloning, Maniatis, Cold Spring-harbor, 2^{nd} edition, pp E3 to E4 [8].

The methods for analyzing labeled products may thus consist in extracting the polynucleotides from these products, detecting the code carried by the recognition polynucleotides, in referring to the database for recognizing target polynucleotides, thus, decrypting the code carried by the target polynucleotides, then in exploiting this information in order to find the target polynucleotides amongst the plurality of target polynucleotides, including the decoy polynucleotides, then in detecting the presence of target polynucleotides, which is a feature of the labeled product (in the case of detection of the labeling) or to conclude that a product is a counterfeit (in the case of absence of target polynucleotides, or labeling that is not in accordance with the targets/products database).

According to a particular embodiment of the present invention, the detection specific to the step of analysis may comprise for example the following successive steps of:
(i) placing in contact the plurality of polynucleotides with a solid support whereon probe sequences are fixed, these probe sequences being complementary to one of the said ends of said, at least one, target polynucleotide of the plurality of polynucleotides of the labeling of the product, the placing in contact allowing the target polynucleotides to be fixed on the support by hybridization with the complementary probe sequences fixed on the support;
(ii) eliminating the polynucleotides non hybridized by step (i); and
(iii) detecting the presence on the support of the target polynucleotides.

In the present invention, the detection carried out at step (iii) may be achieved by an adapted specific means, for example it may relate to a detection using a fluorescent molecule, or a detection using a luminescent molecule, or a detection using an enzyme whereof the reaction product may be colored, or a detection using an enzyme whereof the catalyzed reaction is exothermic, or a detection using an enzyme whereof the catalyzed reaction releases light, or a detection using a protein specific to target polynucleotides, such as for example an antibody, an enzyme.

According to a particular embodiment, the specific detection may further comprise between steps (i) and (ii), a step of capturing the plurality of polynucleotides on a support, by at least a specific capturing system of the target polynucleotide(s), a system whereon is fixed, the at least one, target polynucleotide of the plurality of polynucleotides of the labeling of the product.

When the labeling according to the invention comprises recognition polynucleotides, the method of this particular embodiment may further comprise, before step (i), a step (x) of identifying these recognition polynucleotides and a step (y) of choice of a solid support, according to the identified recognition polynucleotides, solid support selected as it comprises the probe sequences complementary to identified target sequences thanks to the recognition polynucleotides.

This method of detection may advantageously be used with a marker according to the invention which comprises target polynucleotides having a constant end and a variable end. These target polynucleotides are defined above. According to the invention, the polynucleotides complementary to target polynucleotides, called probe sequences, may be fixed on the solid support by any means known by the skilled person. These detection techniques on support and the types of usable supports in the present invention are for example described in Molecular Cloning, Maniatis, Cold Spring-Harbor, 2^{nd} edition, pp 9.47 to 9.57 [9]. For example, the fixing of the probe sequences on the support may be carried out by means of a biotin/streptavidin connection, the probe being coupled to a biotin molecule and the support exhibiting streptavidin molecules. For example, the fixing of the probe sequences may also be achieved by formation of covalent linkages to a charged nylon membrane, said membrane forming the solid support. These techniques, usable in the present invention, are for example those described in publications [10], [11] and [12]. In other words, the polynucleotides complementary to the target polynucleotides are examples of capturing systems specific to the sought target polynucleotide(s).

According to the invention, the target polynucleotides fixed on the support by hybridization to the probe sequences may be detected by any appropriate means known by the skilled person. It may be for example a detection by means of polynucleotides marked by a labeling agent and complementary to the other end of the target polynucleotides, the labeling agent being able to be selected from the group comprising a fluorochrome, a colloidal gold particle and an enzyme.

This detection mode on solid support allows for an easy, reproducible and immediate detection, of the marker of the present invention. It may be advantageously used in the present invention.

The detection method of the invention may further comprise a step of comparison of the results of the step of analysis of the target polynucleotides with the contents of a database which enables to identify the target polynucleotides, and analysis of the target polynucleotides which enables to identify and authenticate said product, and which can also enable to determine the origin of said product. In other words, the database and decrypting of the code carried by the coding polynucleotides enable to identify a counterfeit from an original product.

It is by finding, using the technique of analysis used, the target polynucleotides contained in a labeled product that the information pertaining to this product may be found. The absence of the target polynucleotides which should be present in a tested product indicated a possible counterfeiting of the product. The detection of several different markers according to the invention or recognition polynucleotides in a same product may indicate that it is the result of an abnormal initial mixture.

During the manufacturing steps of the product to be labeled, the manufacturer user of a marker in accordance with the present invention may associate to the identifier of the container of the batch of markers, references pertaining for example to a production batch labeled using this batch of markers. This association may be carried out for example in a database identical to or separated from the previous databases. The information entered into this database and pertaining to the production batch are preferably sufficient, depending on the information system implemented at this user's, to enable to trace this batch unequivocally.

When a product sample suspected of being a counterfeit is analyzed, the detection of identified polynucleotide sequences may be compared with the information entered in the databases during the production and delivery of markers.

Counterfeiting is for example characterized if no marker is identified. Counterfeiting may also be highlighted if at least one marker in the batch of markers having partially been revealed and whereof the precise composition has been obtained by interrogation of the database is missing. Counterfeiting may still be characterized if the integrality of the markers, which should appear in the batch of markers, is present but the tested goods are not from the manufacturer having taken the order for the batch of markers which has just been revealed.

In the case of misappropriation of the distribution channels or parallel markets, the products are authentic and the batches of revealed markers actually correspond to batches of markers delivered to the manufacturer of the tested product. The method of the invention enables to obtain upon request from the manufacture database markers of the identifier of the batch of markers. As far as its traceability system allows, this identifier advantageously enables the manufacturer to compare the theoretical assignment of the labeled production batch to one of his clients with the real assignment noticed during the sampling of the product suspected of parallelism. If the theoretical assignment of the products does not correspond to the real assignment, there may be misappropriation of the distribution channel.

Other advantages may further become apparent to the skilled person upon reading the examples below, illustrated by the accompanying drawings, given by way of illustration.

### BRIEF DESCRIPTION OF THE DRAWINGS

The accompanying FIG. 1 represents a cleavage site of NbBpu 10I.

FIG. 2 represents an addressing of probes on the microarray.

FIG. 3 represents emission and absorption spectrums of Cy5.

The accompanying FIG. 4 represents a method for detecting markers according to the present invention on a support: markers M1 and M2 are present in a mixture. They establish a “bridge” between the probe fixed on the microarray, and the Universal probes: the signal (fluorescence of Cy5) is thus detected.

The accompanying FIG. 5 shows a detection of markers with the NanoChip workstation (registered trademark) (Nanogen Inc.).

The accompanying FIG. 6 shows a principle of coupled labeling.

The accompanying FIG. 8 represents a diagram of detection of a labeling according to the invention and authentication of a labeled product according to the invention.

### EXAMPLES

### Example 1

### Manufacture of a Labeling According to the Method of the Invention and Labeling of a Product

In this example, the target and decoy polynucleotides used are single-stranded deoxyribonucleic acid sequences of a size of 28 nucleotides. The encoding polynucleotide is a circularized DNA sequence, of a size of 4.3 Kilobases (kb).

The labeled product is a perfumed solution: J'Adore perfume (registered trademark, Christian Dior perfumes.)

Three labelings are achieved, illustrating three possible hypothetical cases during the authentication method: an example where the product is authenticated, as well as two examples where the product is not authenticated.

### I.A Design of the Target Markers and the Decoy Polynucleotides

1.a.1 Principle

Ten polynucleotides of single-stranded deoxyribonucleic acid are generated in the following manner: The ten 5′ nucleotides are all identical, defined by the user. It is about the GCAACTCCAG sequence. The eighteen 3′ nucleotides are then generated using the algorithm exhibited in the “Statement of the invention”, by using the following parameters: a length of words equal to 18 nucleotides, five G nucleotides, five C nucleotides, four A nucleotides and four T nucleotides.

Each new polynucleotide is then generated randomly, such that it contains this determined number of each of the bases. An alignment score between this new polynucleotide and the set of already-validated polynucleotides, as well as the polynucleotides resulting from the concatenation of each of the polynucleotides two by two in this set, this step need not take place if it is about the first polynucleotide, is calculated according to the Smith and Waterman algorithm, with the following parameters:
Score of an AT:1 match
Score of GC: 1.5 match
Mismatch penalty: −3
Gap penalty: −2

The minimum selected score is 3, which signifies that is the new polynucleotide is aligned with a polynucleotide from the set, or a concatenation of two polynucleotides from the set with a score higher than 3, it is ruled out. Otherwise, it is validated then added to the set of polynucleotides. This step is repeated until ten polynucleotides are obtained.

The table 1.1 shows the list of these markers.

TABLE 1.1
Sequence of markers

| | |
|---|---|
| Primer-1: | 5′-GCAACTCCAGGCACTCCATGAGTCATGG-3′ |
| | (SEQ ID NO:: 1) |
| Primer-2: | 5′-GCAACTCCAGGTGGCGACTCATACGTCA-3′ |
| | (SEQ ID NO:: 2) |
| Primer-3: | 5′-GCAACTCCAGCTCAGGGGGACTCTATCA-3′ |
| | (SEQ ID NO:: 3) |
| Primer-4: | 5′-GCAACTCCAGGCTCTAGGGCAAGTCTCA-3′ |
| | (SEQ ID NO:: 4) |
| Primer-5: | 5′-GCAACTCCAGGCAGACTCTGGATCTCAG-3′ |
| | (SEQ ID NO:: 5) |
| Primer-6: | 5′-GCAACTCCAGGCAGCATGAGGTCTCATC-3′ |
| | (SEQ ID NO:: 6) |
| Primer-7: | 5′-GCAACTCCAGGCAGCAGGAGTCTCATTC-3′ |
| | (SEQ ID NO:: 7) |
| Primer-8: | 5′-GCAACTCCAGGGTGGCTCAGCAATACTC-3′ |
| | (SEQ ID NO:: 8) |
| Primer-9: | 5′-GCAACTCCAGCTCAGGGCAGTGATCTCA-3′ |
| | (SEQ ID NO:: 9) |
| Primer-10: | 5′-GCAACTCCAGGGGGGCACACATTCTATC-3′ |
| | (SEQ ID NO:: 10) |

The first five polynucleotides (Primer-1 to Primer-5) are considered as target polynucleotides. The last five (Primer-6 to Primer-10) are considered as decoy polynucleotides. These markers are then injected to a final concentration of 10-12 moles per dm³ in the perfume, at the same time as the encoding marker.

1.a.2 Material
Synthesis polynucleotides (Eurofins MWG GmbH), 100 pmoles/μL
H₂O deionized, nuclease-free

1.a.3 Methods

The ten polynucleotides are mixed in three pre-labeling solutions, diluted in deionized nuclease-free water, at a final concentration of 10⁻⁹ moles of markers per liter, per marker (namely 5.10⁻⁹ moles/L of total markers). For each solution, each marker is pre-diluted twice at 1/100 in nuclease-free water (1 μL using a P10 pipette, in 99 μL using a P100 pipette) namely an intermediate dilution at 1/10000. Then, 10 μL of each intermediate dilution are added in a tube of 1.5 mL (Eppendorf, registered trademark), then the mixture is completed at 100 μL by addition of 50 μL of water (using a P100 pipette). The table 1.2 exhibits the three pre-labeling solutions (A, B, C) each containing a combination of five target polynucleotides selected among ten possible ones either target or decoy.

TABLE 1.2
Combinations of markers

| | A | B | C |
|---|---|---|---|
| Primer-1 | x | | x |
| Primer-2 | x | | x |
| Primer-3 | | x | x |
| Primer-4 | | x | x |
| Primer-5 | | x | x |
| Primer-6 | | x | |
| Primer-7 | | x | |

### I.B. Design of Encoding Polynucleotides

1.b.1 Principle

The encoding polynucleotide is a single-stranded, circular nucleic acid sequence, of a length equal to 4.3 Kilo bases (Kb). It is, for the example, synthesized based on a plasmid pBR322. This polynucleotide contains a specific sequence of consecutive nucleotides (A, T, G, C), known by the user of the invention. It constitutes a unique code enabling the user thereafter to know which combination of target markers is in theory present in the labeled product.

In this example, the coding sequence is a portion of 20 nucleotides, located exactly at 50 bases upstream (side 5′) of a known and universal sequence that remains constant whatever the encoding sequence, and whereof the sequence is: 5′-CTGTAAGCGGATGCC-3′ (SEQ ID NO: 11). The user has a table associative enabling him/her to make the connection between the coding sequence of the coding polynucleotide, and the combination of target polynucleotides expected in the labeled product.

| Coding sequence carried by | Combination of expected |
|---|---|
| the coding polynucleotide | target polynucleotides |
| 5′-CCTCGCGCGTTTCGGTGATG-3′ | 1.2 |
| (SEQ ID NO:: 12) | |

The plasmid is first digested by the restriction enzyme Nb.Bpu10I, which cuts a single strnd of the DNA molecule, recognizing the restriction site represented on FIG. 1: cutting site of NbBpu 10I. The polynucletoide is then digested a second time by the exonuclease III, liberating the nucleotides from the free 3′hydroxyl end of the cut strand by Nb.Bpu10I, being circular, the strand which has not been digested by Nb.Bpu10I is thus spared.

1.b.2 Material
Plasmid pBR322, Invitrogen (registered trademark)
NbBpu 10I. (20 U) reference: Fermentas, #ER1681
×R 10 Buffer (buffer Nb.Bpu 10I) reference #BR5, (Fermentas)
Exonuclease III (1200 U) reference #ENO191, (Fermentas)
Reaction buffer for Exonuclease III; reference #ENO191, (Fermentas)
UltraPure Phenol (trademark) Buffer-Saturated Phenol, Invitrogen.
Chloroform/amyl alcohol (24:1)
3 M Sodium acetate
95%, 75% Frozen ethanol
Deionized nuclease-free water.

1.b.3 Methods

Digestion

In a tube of 1.5 mL (Eppendorf, registered trademark), add the plasmid (5 μg namely 20 μL), the ten times concentrated reaction buffer (40 μL) using a pipette P100 (Gilson Pipetman, registered trademark) the water (399 μL) using a pipette P1000 (Gilson Pipetman, registered trademark) as well as the enzyme (1 μL) using a pipette P10 (Gilson Pipetman, registered trademark).

TABLE 1.3
digestion of pBR322

| | |
|---|---|
| Plasmid | 20 μL |
| ×10 Buffer R | 40 μL |
| Nb.Bpu101 (20U) | 1 μL |
| H₂O | 339 μL |

Vortex using a VTX-400 vortex (Labo Moderne) then incubate the tube 1 hour at 37° C. Extract the DNA with Phenol-chloroform according to the following protocol:
Add in the tube ½ volume of phenol (200 μL) and ½ volume of chloroform (200 μL) using a pipette P200 (Gilson Pipetman, registered trademark), then vortex for 10 seconds. Then centrifuge for 5 minutes at 10 000 rcf (in a centrifuge with 1.5 mL tubes “Centrifuge 5415 R”, Eppendorf, registered trademark).
Transfer the acqueous phase in a new 1.5 mL tube (Eppendorf, registered trademark) and add 1 volume (400 μL) of chloroform. Vortex and centrifuge for 5 minutes at 10 000 rcf. Repeat this step 5 times.
Transfer the aqueous phase into a new 1.5 mL tube, then add 1/10 volume of 3M sodium acetate (40 μL, using a P20) and 2.5 volumes (1000 μL using a P1000) of frozen ethanol. Blend then incubate for 1 hour at −20° C.
Centrifuge for 10 minutes at 10 000 rcf. Throw out the supernatant and carefully wash the pellet with 200 μL of 75° frozen ethanol. Then dry the pellet in the open air. Take it back in 50 μL (using a pipette P100) of deionized nuclease-free water.

Treatment with Exonuclease

In a 1.5 mL tube (Eppendorf) containing the 50 μL extract, add using pipettes p100, p20 and P200 the reagents according to table 1.4.

TABLE 1.4
Linearization of pBR322

| | |
|---|---|
| Reaction buffer for ExoIII | 25 μL |
| Exonuclease III (1200 U) | 6 μL |
| H₂O, Nuclease-free | 119 μL |

Mix the tube then incubate for 10 minutes at 30° C. Stop the reaction by heating for 10 minutes at 70° C.
Extract the DNA with phenol Chloroform as described in step 1.b.3, precipitate the DNA as described in step 1.b.3 then take up the polynucleotides in 20 μL of demineralized nuclease-free water.

The markers taken up in 20 μL are dosed using a Nanodrop spectophotometer (registered trademark). The concentration is then brought back to 10-9 mol/L by adding an adequate volume of demineralized nuclease-free water using a P10.

### I.C. Labeling the Solutions

Three perfumed J'Adore solutions (registered trademark, Christian Dior perfumes) are thus labeled. The polynucleotides are injected into the mass of products, at a final concentration of 10⁻¹² M for the target and decoy polynucleotides, and 10⁻¹² M for the coding polynucleotides.

The first solution is labeled with the pre-labeling solution A, containing the target polynucleotides 1 and 2 as well as the decoy polynucleotides 8, 9, 10 and the coding polynucleotide. This solution corresponds to a normal labeling, where the coding polynucleotide is present, and or the combination of present target polynucleotides actually correspond to the information carried by the coding polynucleotide.

The second solution is labeled with the pre-labeling solution B, containing the target polynucleotides 3, 4, 5 as well as the decoy polynucleotides 6 and 7 and the coding polynucleotide. This solution serves as an example for incoherent labeling: although the coding polynucleotide is present, the combination of target polynucleotides does not correspond to the information it contains.

The third solution is labeled with the pre-labeling solution C, containing the target polynucleotides 1, 2, 3, 4 and 5, no decoy polynucleotide and no coding polynucleotide. This solution also serves as an example for incoherent labeling: on the one hand it does not comprise coding polynucleotides, and on the other hand, although it has the target polynucleotides 1 and 2 of normal labeling, it contains other unexpected polynucleotides.

After labeling, the solutions are stored at ambient temperature or at 4° C.

### I.D Extraction of Polynucleotides

Before starting the identification of the coding and target polynucleotides, it is advised to extract the markers from the alcohol medium constituting the labeled product (perfume).

1.d.1 Principle

The markers are extracted from their alcohol medium (perfume), then recovered in an aqueous medium with the purpose of then using the molecular biology identification techniques. The extraction must hence, preferably have a high yield (recover a maximum number of markers, ideally: a 100% yield) but it must also preferably rid the markers of all “polluting” substances that could interfere with the detection techniques.

1.d.2 Material
Phenol: UltraPure (trademark) Buffer-Saturated Phenol, invitrogen.
chloroform/amyl alcohol (24:1)
3 M sodium acetate
95% frozen ethanol (Carlo Erba Rectapur), 75%
deionized nuclease-free water.

1.d.3 Methods

The technique used is that of the phenol-chloroform extraction. For the example, the markers are extracted from 500 μL of labeled perfume.
At 500 μL of labeled perfume in a 2 mL tube (Eppendorf, registered trademark), add ½ volumes of phenol namely 250 μL using a pipette P1000 (Gilson Pipetman, registered trademark), then ½ volumes of chloroform namely 250 μL using a P1000 (Gilson Pipetman, registered trademark), and ½ volume of water namely 250 μL using a P1000 (Gilson Pipetman, registered trademark) then vortex for 10 seconds. Centrifuge for 5 minutes at 10 000 rcf (in a “Centrifuge 5415 R”, centrifuge, Eppendorf, registered trademark).
Transfer the aqueous phase to a new 2 mL tube (Eppendorf, registered trademark), and add 1 volume of chloroform. Vortex and centrifuge for 5 minutes at 10 000 rcf. Repeat this step 5 times.
Transfer the aqueous phase (above) to a new tube, then add 1/10 of volume of 3M sodium acetate (namely 50 μL using a pipette P100) and 2.5 volumes of frozen ethanol (namely 1250 μL, added in two parts using a P1000). Blend then incubate for 1 hour at −20° C.
Centrifuge for 10 minutes at 10 000 rcf. Throw out the supernatant and carefully wash the pellet with 200 μL of 75% frozen ethanol. Then dry the pellet. Take it up in 20 μL of nuclease-free deionized water, using a pipette P20.

### I.E Detection of the Coding Polynucleotides

1.e.1 Principle

The information carried by the coding polynucleotides, pertaining to the target polynucleotides, is read in the nucleic sequence of these first (by a sequencing technique). It then enables the user of the invention to know the exact nature of the target polynucleotides carrying the authentication information of the product by referring to the table associative in 1.b.1.

1.e.2 Material
Universal sense primer (Eurofins MWG GmbH), 100 pmoles/μL

| | |
|---|---|
| 5′-GGCATCCGCTTACAG-3′ | (SEQ ID NO: 13) |

Big Dye (registered trademark) Terminator V3.1 (Applied Biosystems)
Template: solution of extracted polynucleotides.
Deionized nuclease-free H₂O.

1.e.3 Methods

The sequencing reaction is carried out based on the solution of extracted markers in a tube of 200 μL (Eppendorf, registered trademark), according to the protocol summarized in table 1.5

TABLE 1.5
Sequencing reaction

| Reagent | 1 sequence | |
|---|---|---|
| Big Dye (registered | 2 | μL |
| trademark) | | |
| Polynucleotides | 5 | μL |
| (template) | | |
| Primer 10 pM | 1.6 | μL |
| H₂O | q.s 10 | μL |

The reactions are then launched on an apparatus GeneAmp PCR System 9700 (Applied Biosystem) while respecting the following cycles:

| | | | | |
|---|---|---|---|---|
| −96° C. | 1′ | | | |
| −96° C. | 10″ | \| | | |
| −50° C. | 5″ | \| | => | 25 cycles |
| −60° C. | 4′ | \| | | |

After column-based purification (Qiagen, registered trademark), the reactions are then sequenced on a 16-capillary sequencer AB13100 (Applied Biosystem). The analysis of the results thus enables the user of the invention to read the nucleotide sequences of interest, and to carry out the correlation between the read sequences and the target polynucleotides theoretically present in the labeled product.

### I.F Detection of the Target Nucleotides

1.f.1 Principles

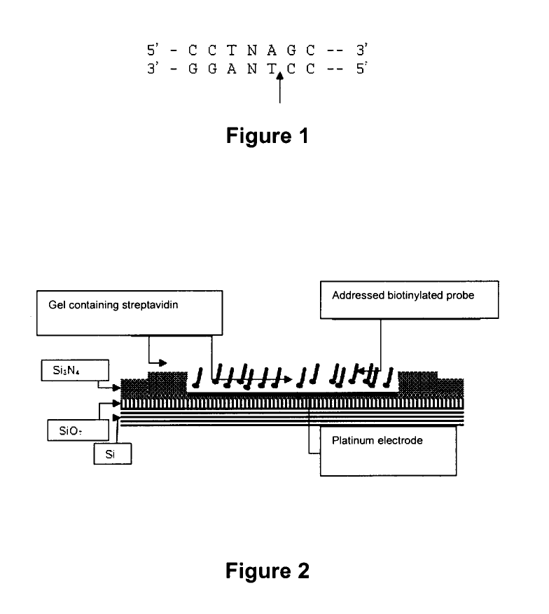

A support (or DNA microarray), is used to detect the presence of target polynucleotides. This support exhibits a battery of several probes, fixed covalently, and exact reverse complements of the markers 3′ variable regions. Accompanying FIG. 2 represents the position of the probes on the chip.

When the extracted solution, containing putative markers, is put in contact with this support, the present markers hybridize with their reverse complements on the chip. After washing, the support if then place in contact with a solution containing probes (polynucleotides) coupled to a fluorophore (Cy5), whereof the sequence is exact reverse complement of the markers 5′ Universal region.

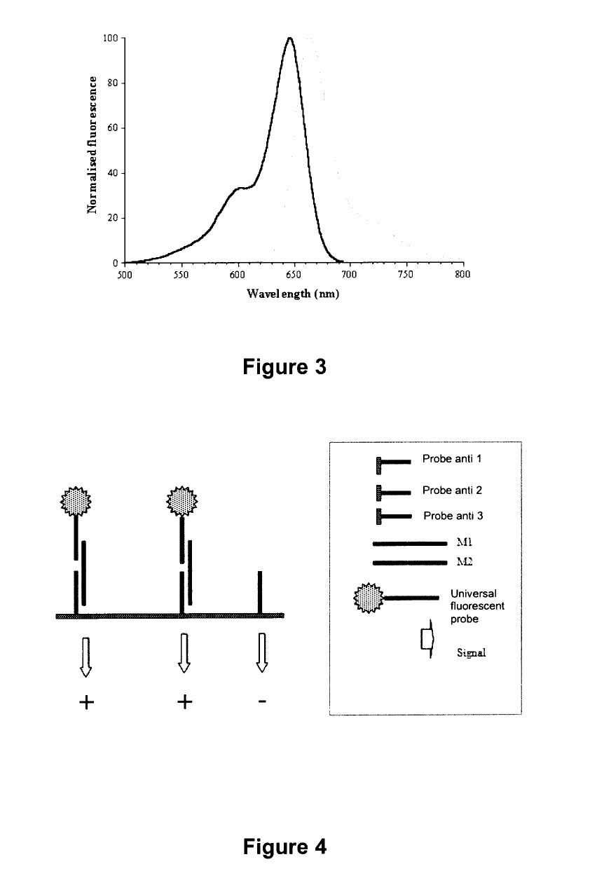

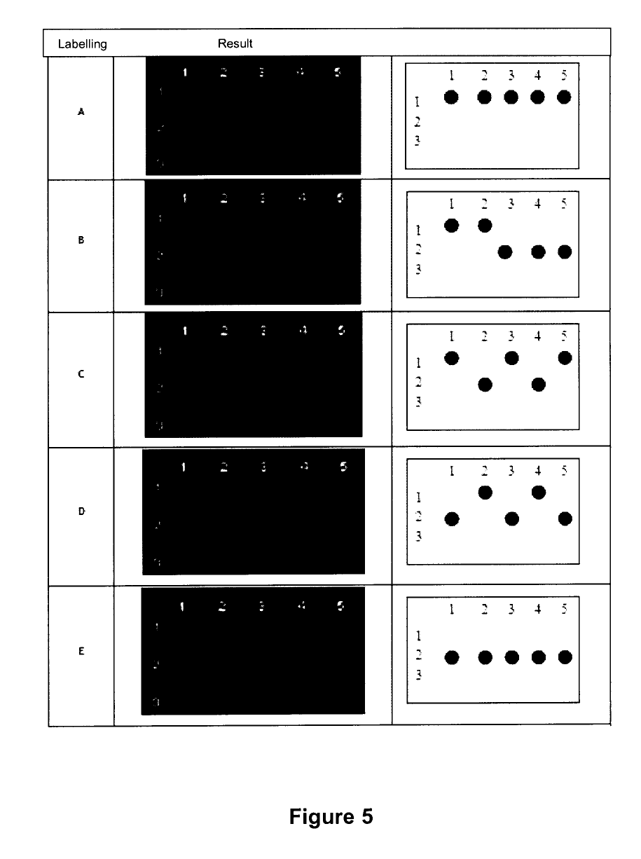

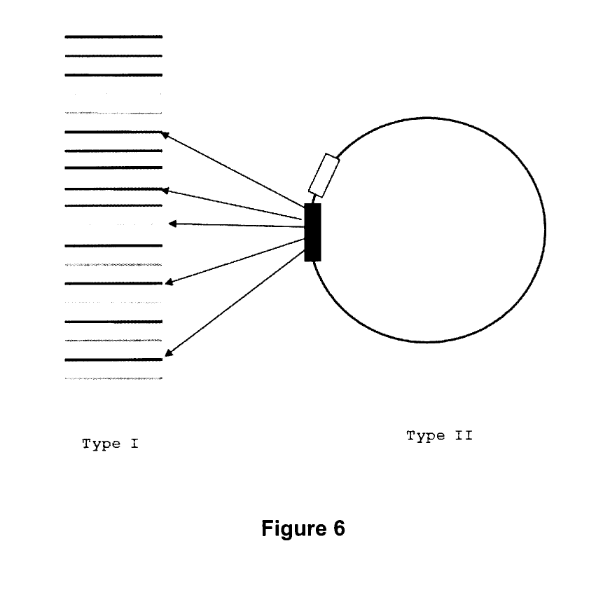

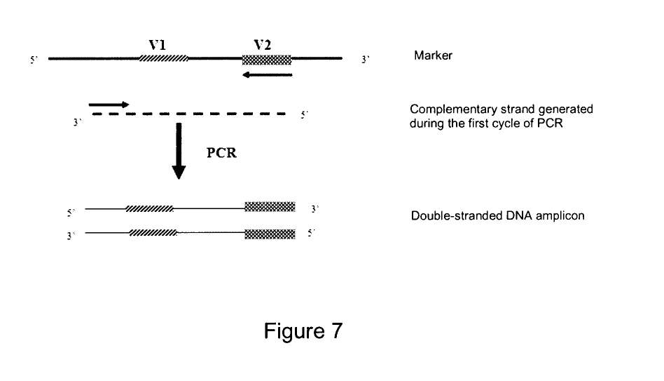

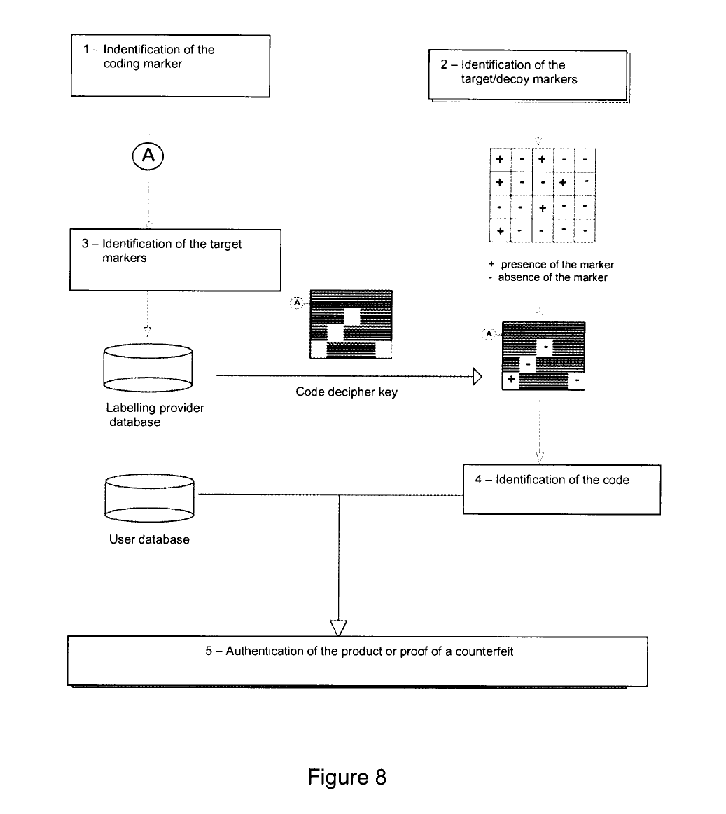

Accompanying FIG. 3 represents the emission (667 nm) and excitation (650 nm) spectrum of Cy5.

These probes fix themselves thereto, and are detected thereafter. Their presence enables to detect the presence of the corresponding target polynucleotides in the initial solution. Accompanying FIG. 4 represents the detection principle of the markers.

1.f.2 Material
reverse complement probes of the markers specific regions, (Eurofins MWG GmbH), 5′ extended with an arm of 20 bases and biotinylated.

Probe anti-1
(SEQ ID NO: 14)
5′ (B)-TGGATCCCGCACACGACTGACCATGACTCATGGAGTGC 3′
Probe anti-2
(SEQ ID NO: 15)
5′ (B)-TGGATCCCGCACACGACTGATGACGTATGAGTCGCCAC 3′
Probe anti-3
(SEQ ID NO: 16)
5′ (B)-TGGATCCCGCACACGACTGATGATAGAGTCCCCCTGAG 3′
Probe anti-4
(SEQ ID NO: 17)
5′ (B)-TGGATCCCGCACACGACTGATGAGACTTGCCCTAGAGC 3′
Probe anti-5
(SEQ ID NO: 18)
5′ (B)-TGGATCCCGCACACGACTGACTGAGATCCAGAGTCTGC 3′
Probe anti-6
(SEQ ID NO: 19)
5′ (B)-TGGATCCCGCACACGACTGAGATGAGACCTCATGCTGC 3′
Probe anti-7
(SEQ ID NO: 20)
5′ (B)-TGGATCCCGCACACGACTGAGAATGAGACTCCTGCTGC 3′
Probe anti-8
(SEQ ID NO: 21)
5′ (B)-TGGATCCCGCACACGACTGAGAGTATTGCTGAGCCACC 3′
Probe anti-9
(SEQ ID NO: 22)
5′ (B)-TGGATCCCGCACACGACTGATGAGATCACTGCCCTGAG 3′
Probe anti-10
(SEQ ID NO: 23)
5′ (B)-TGGATCCCGCACACGACTGAGATAGAATGTGTGCCCCC 3′

Polynucleotides reverse complements of the markers 5′ Universal region, coupled to a fluorophore (Cy5) in 3′; (Eurofins MWG GmbH):

| | |
|---|---|
| UnivFluo 5′ CTGGAGTTGC-(CY5) 3′ | (SEQ ID NO: 24) |

Solution of extracted markers
Chip and cartridge for Nanogen automation (trademark) spotted with the complements of the variable regions of the probes (NanoChip (registered trademark) Electronic Microarray, 100-Site NanoChip (registered trademark) Cartridge)
NanoChip workstation (registered trademark) Molecular biology Workstation (NanoChip (registered trademark) Reader, NanoChip (registered trademark) Loader)
L-histidine (Invitrogen, reference: 0955061IX)
Multiscreen Filtration system (Millipore (registered trademark))
High salinity buffer (sodium phosphate 500 mM, sodium chloride 500 mM, Nanogen (registered trademark))
Low salinity buffer (sodium phosphate 50 mM, Nanogen (registered trademark))
NaOH 0.1 M

1.f.3 Methods

Preparation of the Cartridge

The probes complementary to the specific regions of the markers are purified by the Multiscreen filtration system (by Millipore (registered trademark)) then taken up in 60 μL of buffer L-histidine 50 mM. They are then transferred to the cartridge. Each one of them is then addressed to a specific site on the cartridge during a period of 120 seconds (protocol managed by the workstation). The biotin in 5′ is fixed on the streptavidin of the support.

TABLE 1.6
Probe spotting map

| | 1 | 2 | 3 | 4 | 5 |
|---|---|---|---|---|---|
| 1 | Anti 1 | Anti 2 | Anti 3 | Anti 4 | Anti 5 |
| 2 | Anti 6 | Anti 7 | Anti 8 | Anti 9 | Anti 10 |

Hybridization of the Markers

Rinse the cartridge twice with the high salinity buffer (75 μL). Prepare a mixture containing 5 μL of extract (markers), as well as fluorescent probes with a final concentration of 0.5 μM in a high salinity buffer (s.a.t 100 μL). Take a sample of 75 μL of this mixture from the cartridge then incubate it for three minutes at ambient temperature. Empty the cartridge then rinse twice with 75 μL of the high salinity buffer. Add 75 μL of high salinity buffer at the end of the operation. Then start-up the apparatus by using the standard protocol of the Nanogen workstation (registered trademark). Regenerate the cartridge which may be re-used.

### I.G Exploitation of the Results and Interpretation

The analysis of the three labeled products in this first example (cf. FIG. 5) enables to simulate three possible configurations (among others) during product authentication steps.

In the first and second labeled products, the user was able to detect the presence of the coding polynucleotide. The actual presence of this polynucleotide indicates that the product is liable to be authentic. As regards to the third product, the total absence of detection of the coding polynucleotide indicated that the product is not labeled: hence, it is not authenticated.

After reading the information carried by the coding polynucleotide present in the first and second product, the user refers to the associative table which indicates that the code of the coding polynucleotide corresponds to the presence of the target polynucleotides 1 and 2 (it does not matter if there are decoy polynucleotides present). These polynucleotides are properly detected in the first labeled solution, and only these two polynucleotides: the first solution may be authenticated. As to the second solution, the unexpected presence of the target polynucleotides 3, 4 and 5 does not allow to authenticate the product.

### Example 2

### Labeling a Cosmetic Product According to the Method of the Invention

This example illustrates a labeling using several single-stranded deoxyribonucleic acid polynucleotides, injected in a skin cream, and detected according to the technique exhibited in the example 1.

II.A. Injection of the Polynucleotides into the Cream

2.a.1 Principle

The markers are first prepared from nuclease-free distilled water, then incorporated in the cream at a final concentration of 10⁻¹² Mole per caplet of dm³ of cream for the target and decoy markers, and at a concentration of 10⁻¹⁴ moles per dm³ of cream for the coding markers.

2.a.2 Material
Synthesis polynucleotides (Eurofins MWG GmbH), 100 pmol/μL
Circular single-stranded deoxyribonucleic acid (coding markers)
H₂O deionized, nuclease-free
Thermal cream Fix 2 (registered trademark), VICHY Laboratoires

2.a.3 Methods

A preliminary mixture of coding target and decoy polynucleotides, is injected using a pipette P10 (Gilson Pipetman, registered trademark) in samples of 1 cm³ of Thermal Fix cream (registered trademark), leading to a final concentration of 10⁻¹² moles of marker per dm3 for target and decoy markers, and a final concentration of 10¹⁴ moles per dm³ for the coding polynucleotides. The samples are then kept for their identification.

### II.B. Extraction and Detection of the Markers

The markers are detected in the same manner as example 1, except for the polynucleotide extraction step.

2.b.1 Extraction Principle

The markers are extracted from the cream by breaking up the emulsion and recuperating the aqueous phase. A high temperature (higher than 80° C.) is sufficient for reducing the emulsion constituting the cream and thus separating the aqueous and lipid phases. The markers, very polar, are found in the aqueous phase wherefrom they are extracted.

2.b.2 Material
Thermal Fix cream (registered trademark) (Vichy) Labeled
Heating block or water bath.
H20 demineralized, nuclease-free.

2.b.3 Method

The labeled cream is heated for 15 minutes at a temperature of 95° C. then centrifuged for 5 minutes at 10 000 rcf. The aqueous phase is recuperated, and is used for detecting the markers.

The polynucleotides may then be detected according to the method described in example 1.

### Example 3

### Labeling a Spirit According to the Method of the Invention

This example illustrates a labeling technique of a spirit using a mixture of target, coding and decoy polynucleotides. The first type of marker (target polynucleotides) is constituted of a pool of 20 single-stranded deoxyribonucleic acids of a size of 20 bases. The second marker is a circular single-stranded nucleic acid, of a size of 1000 bases, whereof the sequence contains the research instructions of the target markers. Thus, this sequence enables to know which target markers, among the 20, are significant for the coding of the spirit, the other markers being decoys added semi-randomly.

### III.A. Design of the Markers and Labeling

3.a.1 Detailed Principle of the Labeling Technique

Twenty single-stranded deoxyribonucleic acid markers are generated thanks to the algorithm exhibited in the description of the invention. These markers differ from each other, do not auto-hybridize, and are not liable to hybridize with each other. They constitute the target polynucleotides.

The coding polynucleotide is a circular deoxyribonucleic acid of 1000 bases. The sequence of this nucleic acid contains, at a given point, a cassette containing the combination of the target polynucleotides to search for in the product.

The accompanying FIG. 6 exhibits the principle of coupled labeling: the coding marker, circular, contains a site of general information, as well as a site enabling to know which of the target markers, also present in the mixture, carry the code information (the others being merely decoys).

Thus, only the combination of target markers designated by the coding marker is significant for the authentication, the other markers merely being decoys.

The sequence of the coding marker hence contains a coding box, whereof the position is hidden. The reading of this sequence enables the user to know which markers are to be sought in the pool of target markers via cross checking in a correspondence table.

The combination of target markers enables to identify the product in a unitary manner: one single possible combination for a possible product. In the coding marker, downstream from the coding cassette for the combination of the target markers to be sought, a second code sequence for general information pertaining to the product, such as the batch, the manufacture year, etc.

These markers are hence injected in the spirit to mark. The detection then unfolds in two phases. The first consists in detecting the specific sequences of the coding marker. The first information which is drawn from it is the following:
Product authentication. In case of a gross counterfeiting, no coding marker is present.
Information on the product: batch N°, manufacture date, . . .
Unique combination of target markers to be sought for a more thorough detection.

If this first authentication is not considered to be sufficient, a second one is carried out on the target markers. This second authentication enables to deduce the following information:
Gross counterfeiting in case of absence of target markers or wrong combination.
Product from a mixture if other markers are detected in addition to those provided by the coding marker.
A precise authentication of the product by consulting a database.

3.a.2. Material
Synthesis polynucleotides (Eurofins MWG GmbH), 100 pmoles/μL (target markers)
Moët et Chandon champagne (registered trademark), Brut Imperial
Circular single-stranded deoxyribonucleic acids (coding markers)
Deionized nuclease-free water

3.a.3 Method

a. Target Markers

Target markers are constituted of 20 different polynucleotides, obtained by chemical synthesis (Eurofins MWG GmbH). This example is carried out with the labeling of 5 different products. In each of these products, the 20 markers may be used:

| (SEQ ID NO: 25) | |
|---|---|
| Primer-1: | 5′-AGTCGAGAGCCGATTCCGCT-3′ |
| (SEQ ID NO: 26) | |
| Primer-2: | 5′-GTCCGAGCAAAGGCTTCCGT-S′ |
| (SEQ ID NO: 27) | |
| Primer-3: | 5′-AGACCCGTGGGCTCCATTAG-3 1 |
| (SEQ ID NO: 28) | |
| Primer-4: | 5′-CCACCCAGAGGGCTTAGGTTT3 1 |
| (SEQ ID NO: 29) | |
| Primer-5: | 5′-ATCCCACGAGGGTGATCTCG-3 1 |
| (SEQ ID NO: 30) | |
| Primer-6: | 5′-GGAATCCGACCGTGCATGTC-3 1 |
| (SEQ ID NO: 31) | |
| Primer-7: | 5′-CAGAGACGTGACCCGCTGTT-3 1 |
| (SEQ ID NO: 32) | |
| Primer-8: | 5′-GACCCAGGGGTACATTCTCG-3 I |
| (SEQ ID NO: 33) | |
| Primer-9: | 5′-AAACGAGCCCGTTCCGTGTG-3 1 |
| (SEQ ID NO: 34) | |
| Primer-10: | 5′-GGGAGCCCCAGCATTATCGT-3 1 |
| (SEQ ID NO: 35) | |
| Primer-11: | 5′-GGACGTGAACGCATCCGTCT-3 I |
| (SEQ ID NO: 36) | |
| Primer-12: | 5′-GGCTGAAGGCCACTACTCTG-3′ |

| (SEQ ID NO: 37) | |
|---|---|
| Primer-13: | 5′-GTAGGTAGCACACCGTCGCT-3 I |
| (SEQ ID NO: 38) | |
| Primer-14: | 5′-CAGCCAGGAGATGTCCGTCT-3 1 |
| (SEQ ID NO: 39) | |
| Primer-15: | 5′-GTCCCCAGGTGAGATCATCG-3 1 |
| (SEQ ID NO: 40) | |
| Primer-16: | 5′-CGAGGGACCAGCTTCCGTAT-3 I |
| (SEQ ID NO: 41) | |
| Primer-17: | 5′-GCCAGTCGCAGGCATGATTC-3 1 |
| (SEQ ID NO: 42) | |
| Primer-18: | 5′-CGCCAGGGTCTCAGTCGTAA-3 1 |
| (SEQ ID NO: 43) | |
| Primer-19: | 5′-GAGCATAGCCGACGTCTTCG-3 1 |
| (SEQ ID NO: 44) | |
| Primer-20: | 5′-GTAGAGTGACACGTCGCTCC-3 I |

On these 20 target markers, only 10 are actually injected in the products. On these 10 markers, 5 are decoys, and 5 constitute the effective labeling of the product. Only the sequence of the coding marker enables to find which the coding markers are, and which the decoys are. Furthermore, the products 1 to 4 are samples of Moët et Chandon champagne, cuvée impériale 2005 (registered trademark), the product number 5 is a sample of Moët et Chandon champagne, cuvée impériale 2002 (registered trademark).

TABLE 3.1
Labeling products 1 to 5 with the target markers 1 to 20

| | Product 1 | Product 2 | Product 3 | Product 4 | Product 5 |
|---|---|---|---|---|---|
| Primer 1 | — | D | — | L | — |
| Primer 2 | — | D | L | — | — |
| Primer 3 | — | D | — | — | — |
| Primer 4 | L | L | — | — | L |
| Primer 5 | — | D | — | L | — |
| Primer 6 | L | L | L | — | L |
| Primer 7 | — | D | — | — | — |
| Primer 8 | L | L | D | D | L |
| Primer 9 | — | — | — | L | — |
| Primer 10 | L | L | L | — | L |
| Primer 11 | — | — | D | D | — |
| Primer 12 | L | L | D | D | L |
| Primer 13 | — | — | — | L | — |
| Primer 14 | — | — | L | — | — |
| Primer 15 | D | — | — | — | D |
| Primer 16 | D | — | — | L | D |
| Primer 17 | D | — | L | — | D |
| Primer 18 | D | — | D | D | D |
| Primer 20 | D | — | D | D | D |

The table 3.1 illustrates the labeling of the 5 products. The dash indicates that the marker is not injected. The letter L indicates that the marker is injected, and that it serves as signature of the labeling. The letter D signifies that the marker is injected, but is serves as a decoy. Thus, products 1 and 2, although they contain different decoys, they contain the same markers. They have the same signature. On the contrary, products 3 and 4 have the same decoys, but the markers are different. Hence, they have a different signature. Product 5 has the same signature as product 1. Only the coding marker differentiates between the two products.

Pre-diluted at a concentration of 10⁻⁷ moles/L in labeling product (1 μL of initial solution at 10⁻⁴ mol/L diluted in 999 μL of labeling product, then 10 μL of this intermediate solution in 999 μL of labeling product), the markers are injected at the rate of 100 μL (using a Pipette P100) such as to obtain a final concentration of 10⁻⁹ moles/L in each of the products 1 to 5 whereof the final volume is, for example, of 10 mL. The product hence contains 10⁻⁸ moles of markers per liter, namely a total 10⁻¹⁰ moles of markers in 10 mL.

β. Coding Marker

Synthesized according to the protocol of example 1, the coding markers are single-stranded deoxyribonucleic acids of a total length of 1000 bases. Their sequence is the following (SEQ ID NO: 45):

5′CAGAAGAATGCACGCTCTTTAACGCTTCGCCCTAAAATGGGCCATG
ACTATTGAGAATACGATACCTTCCCGCGTTAGCATCCCTTCCCTGATG
CTGGTAGATCTACACCATCTGTACGGGAGATAAGGCTGGCTGTGCGCT
TAGACGGGAACTTGGACCGGAAGAATGCGTACAGCCTTACGCGCATCC
GAGTCGTCACCTACCACACGCTCATGCGCACTTTACGGGTAAAAAGTG
TTAATCGTAACAGTGTCGGGACCACTCCTATGCTAATACCAGCGTGGT
CCAGTGACGTTTTTLACATAGTAGGTGCTCTAATCTTGCAAACCACCG
TTTCATTATCTGTTATTCTCCCTTGCTAATGGCCCGCTCAGCACCGGG
TGTTCCCAGAGGAGAGCTCCCAGCCACGTTGCAGCGAGCGGGCTGTCG
AAAGTATAAAGTTCTAC[labeling1]ACATTCTATGCGTATCATTT
CTCCTACGGATCTGGAGCTAATCCGGTACGCAGCTTACGCACAATGGC
ATAAGCTGTGACGTGGGATAGATAGTACTCTCTACAAACGTACAGAGC
AGTGTTATCGATACCCCCCGCAGCCAATTACTCATAAATCCGACACAG
CAACGCCCATTTTCAGTTTTCGGTATACGTGCGGGCTCCTCATATGTA
TTAACGTTGAGTGACTCTGCAGTCCGATTGGATGTTGGTTCCATCCGC
TATCGTAGAGTCCTATC[labeling2]GAAATCCACAAATCTGTGCG
ATTTGACGTACATTTCGTATCGGCGGTAGTTACGCAAGCTAGCACTAC
CATAGTAGTATCGTTATTCGGGCTTGACAACCTCGAAGGCGTGGGGAA
GAAGCGTCAGTATCTTCGCAAGAAATGAGGAAGAGGTACCAATAAGCG
AATGGGCCCGAAACTACGTCCTCGCAGAGGGCGATCGATGGGCGATTA
GAGTCTGGGGGGTAGTTCAGCACA 3′

Mark 1 codes for the combination of target markers to search for in the product. Mark 2 codes for a generic information such as the cuvee where the samples are taken from. Below (table 3.2), the caption of the labels of type 1.

TABLE 3.2
Coding of target markers according
to the variable sites of the coding markers
First labeling

| | Corresponding |
|---|---|
| Sequence of the Marker | target markers |
| 5′ CGAAATAATCTGCCCGGTCT 3 | Primers 4, 6, 8, 10, 12 |
| (SEQ ID NO: 46) | |
| 5′ACTCGTTTAGGGAAGCTCTA 3′ | Primers 2, 6, 10, 14, 17 |
| (SEQ ID NO: 47) | |
| 5′AGCGCATGATATATAGTACC 3′ | Primers 1, 5, 9, 13, 16 |
| (SEQ ID NO: 48) | |
| 5′GGTACTAAGAGTGGCATTGC 3′ | Primers 3, 12, 13, 14, 15 |
| (SEQ ID NO: 49) | |

TABLE 3.3
Coding of the type of product according to the
secondary information of the coding markers
Second labeling

| Sequence of the Marker | Information |
|---|---|
| 5′ ATTTGGAGGCCCGAATACAA 3′ | Moët et Chandon |
| (SEQ ID NO: 50) | champagne, cuvée |
| | Impériale 2000 |
| 5′ AGCCCCATAAGACGCGCTAA 3′ | Moët et Chandon |
| (SEQ ID NO: 51) | champagne, cuvée |
| | Impériale 2002 |

Thus, a specific coding marker is injected into the 5 products, indicating which the coding target authentication markers are for the information, and indicating, in our example, the vintage from which the sample is taken, at a final concentration of 10⁻¹¹ moles/L according to the technique described in the example 1. They contain the following variable sequences:

TABLE 3.4
summary of the markers of products 1 to 5

| L | LP | Marker 1 | Marker 2 |
|---|---|---|---|
| 1 | 1 | 5′CGAAATAATCTGCCCGGTCT 3′ | 5′ATTTGGAGGCCCGAAT |
| | | (SEQ ID NO: NO 45) | ACAA 3′ |
| | | | (SEQ ID NO: NO 50) |
| 2 | 2 | 5′CGAAATAATCTGCCCGGTCT 3′ | 5′ATTTGGAGGCCCGAAT |
| | | (SEQ ID NO: NO 52) | ACAA 3′ |
| | | | (SEQ ID NO: NO 50) |
| 3 | 3 | 5′ACTCGTTTAGGGAAGCTCTA 3′ | 5′ATTTGGAGGCCCGAAT |
| | | (SEQ ID NO: NO 53) | ACAA 3′ |
| | | | (SEQ ID NO: NO 50) |
| 4 | 4 | 5′AGCGCATGATATATAGTACC 3′ | 5′ATTTGGAGGCCCGAAT |
| | | (SEQ ID NO: NO 48) | ACAA 3′ |
| | | | (SEQ ID NO: NO 50) |
| 5 | 5 | 5′CGAAATAATCTGCCCGGTCT 3′ | 5′AGCCCCATAAGACGCG |
| | | (SEQ ID NO: NO 48) | CTAA 3′ |
| | | | (SEQ ID NO: NO 46) |

L = Labeling
LP = labeled product

### III.B. Extraction of the Markers

The detection is carried out in two steps. The first consists in detecting the coding markers. It enables a first authentication of the products, thanks to the second mark. The reading of the first mark then enables to know the combination of the target markers to search for.

3.b.1 Extraction Principle

The markers are extracted from their environment (here, a champagne wine), then recovered in an aqueous environment with the purpose of being able to use the molecular biology identification techniques afterwards. The extraction should preferably have a high yield (recover a maximum of markers, ideally: a yield of 100%), but it should also rid the markers of any “polluting” substances which may interfere with the detection techniques. Furthermore, it should preferably be efficient for the two types of markers.

3.b.2 Material
UltraPure Phenol (trademark) Buffer-Saturated Phenol, Invitrogen.
chloroform/amyl alcohol (24:1)
sodium acetate 3M
95% Frozen ethanol (Carlo Erba Rectapur), 75%
UltraPure Glycogen, Invitrogen (trademark) 20 μg/μmol
Nuclease-free de-ionized water.

3.b.3 Method

The technique used is that of the extraction with phenol chloroform. For the example, the markers are extracted based on 500 μL of product (Champagne).
In a tube of 2 mL (Eppendorf, registered trademark) containing 500 μL of labeled product, add ½ volumes of phenol (250 μL), ½ volume of chloroform (250 μL) using a pipette P1000, then vortex for 10 seconds. Centrifuge for 5 minutes at 10 000 rcf.
Transfer the aqueous phase to a new tube of 2 mL (Eppendorf, registered trademark), and add 1 volume (500 μL) of chloroform. Vortex and centrifuge for 5 minutes at 10 000 rcf. Repeat this step 5 times.
Transfer the aqueous phase to a new tube, then add 3 μL of glycogen, 1/10 volume (50 μL) of sodium acetate 3M and 2.5 volumes (1250 μL) of frozen ethanol. Blend then incubate for 1 hour at −20° C.
Centrifuge for 10 minutes at 10 000 rcf. Throw away the supernatant and carefully wash the pellet with 200 μL of 75% frozen ethanol. Then dry the pellet. Take it up in 20 μL of nuclease-free de-ionized water.

### III.C. Detection of the Coding Marker

3.c.1 Principles

The coding marker is detected by a chain polymerization technique. Two primers are needed to do this. A first type of primer is complementary to the region located at 5′ of the variable sequence 1. This primer is called Universal as it does not depend on the variability of the target markers (it recognizes a site that is common to all these markers). As to the second primer, it is complementary to the variable sites 2. Thus, as many couples of primers as types of variable sequences are used 2 (here, two couples).

Accompanying FIG. 7 represents the detection of the primary labeling of the coding markers by PCR. This fig. resumes these two types of primers, the template as well as the strand which itself is complementary and which is generated during each first PCR cycle.

For each type of variable sequence 2, a polymerization chain reaction is carried out on the markers extracted from the products. The specific amplification of the coding marker for a couple of primers reveals the presence of a sequence of type 2. The absence of amplification reveals an unlabeled product, hence probably a counterfeit. An amplification of the marker with the wrong couple of primers, or with several coupled of primers reveals trickery as to the product (trickery on Ia cuvee, mixtures, . . . )

3.c.2 Material
Taq Polymerase (Applied Biosystems) AmpliTaq Gold
10 Buffer×PCR Buffer II (Applied Biosystems)
MgCl₂ Solution (25 mM) (Applied Biosystems)
Universal sense primer (Eurofins MWG GmbH), 100 pmol/μL)

| | |
|---|---|
| 5′ ACGTTGCAGCGAGCG 3′ | (SEQ ID NO: 56) |

Antisense primer (cf. variable sequences V2: antisense primers are their reverse complements) (Eurofins MWG GmbH), 100 pmol/μL)
dNTP (2 mM)
Agarose gel (Agarose Electrophesis Grade, Invitrogen, reference: 15510-027)
Tris-borate buffer EDTA (TBE) 0.5×
Ethidium bromide

3.c.3 Methods

In a tube of 200 μL (Eppendorf, registered trademark), add the reagents using a Pipette P2, P10 and P100, according to the table 3.5.

TABLE 3.5
Polymerization chain reaction

| Reagent | Volume (μL) |
|---|---|
| Template: Extraction product | 5 |
| Universal sense primer (10 pM) | 0.5 |
| Specific antisense primer (10 pM) | 0.5 |
| Buffer 10× | 5 |
| dNTP (2 mM) | 2.5 |
| MgCL₂ (25 mM) | 2.5 |
| AmpliTaq Gold (5 U/μL) | 0.5 |
| H₂O q.s 50 μL | 33.5 |

The polymerization chain reactions are thus launched on a GeneAmp PCR System 9700 apparatus (Applied Biosystem) by respecting the following cycles:

| | | | | |
|---|---|---|---|---|
| −94° C. | 5′ | | | |
| −94° C. | 30″ | \| | | |
| −55° C. | 30″ | \| | => | 40 cycles |
| −72° C. | 30″ | \| | | |
| −72° C. | 7′ | | | |

Polymerization chain reaction products are thus deposited on an 0.5% agarose gel prepared in TBE 0.5× and placed to migrate in a buffer TBE 0.5× to 10 V.cm⁻¹. After an adequate migration time, the gel is placed in a bath containing BET, rinsed then visualized under UV rays. For every product, the absence of strip reveals that the marker has not been detected, or that the sequence 2 does not correspond to the type of primer used. A strip (size of 358 bp) corresponds to an amplification, hence on detection of a specific sequence of the coding marker.

For every positive detection, the amplicons are kept for a possible detection of the target markers.

### III.D Detection of the Target Markers

3.d.1 Principle

The target markers are sought in the product for a more thorough detection of the labeling. It is only by reading the coding marker that the target markers may be detected. This detection is carried out on the previously obtained amplicons by polymerization chain reaction. The first step consists in reading the information contained on the coding markers, and hence on the amplicons. This information (sequences 1) enables to know, thanks to a correlation table, which target markers are present in the product whereof the presence carries the labeling information. By knowing this, a detection of the total target markers is achieved, by using a DNA microarray. After revealing this detection, the user is able to know which target markers are present or not in the product, and may compare these results to the results obtained theoretically by reading the information carried on the coding markers.

3.d.2 Material
Universal sense primer (Eurofins MWG GmbH), 100 pmol/μL)

| | |
|---|---|
| 5′ ACGTTGCAGCGAGCG 3′ | (SEQ ID NO: 56) |

Big Dye (registered trademark) Terminator V3.1 (Applied Biosystems)
Template: coding amplicons marker
Nuclease-free de-ionized H₂O.

3.d.3 Methods

The sequence reaction is carried out on each of the amplicons, in a tube of 200 μL (Eppendorf, registered trademark), according to the protocol summarized in the table 3.6.

TABLE 3.6
Sequencing reaction

| Reagent | 1 sequence | |
|---|---|---|
| Big Dye (registered trademark) | 2 | μL |
| Amplicon (template) | 5 | μL |
| Primer 10 pM | 1.6 | μL |
| H₂O | sat. 10 | μL |

The reactions are then launched on a GeneAmp PCR System 9700 apparatus (Applied Biosystem) by respecting the following cycles:

| | | | | |
|---|---|---|---|---|
| −96° C. | 1′ | | | |
| −96° C. | 10″ | \| | | |
| −50° C. | 5″ | \| | => | 25 cycles |
| −60° C. | 4′ | \| | | |

After column-based purification (Qiagen, registered trademark), the reactions are then sequenced on a 16-capillary sequencer ABI3100 (Applied Biosystem).

The variable sequences 1 (or Labels 1) are extracted from the result of these sequences. These sequences enable to find which target markers must be present in the mixture, by consulting the following table (table 3.7):

TABLE 3.7
Coding target markers according to the
variable sites of the coding markers

| | Corresponding |
|---|---|
| Sequence of the Marker | target markers |
| 5′CGAAATAATCTGCCCGGTCT 3′ | Primers 4, 6, 8, 10, 12 |
| (SEQ ID NO: 52) | |
| 5′ACTCGTTTAGGGAAGCTCTA 3′ | Primers 2, 6, 10, 14, 17 |
| (SEQ ID NO: 53) | |
| 5′AGCGCATGATATATAGTACC 3′ | Primers 1, 5, 9, 13, 16 |
| (SEQ ID NO: 54) | |
| 5′GGTACTAAGAGTGGCATTGC 3′ | Primers 3, 12, 13, 14, 15 |
| (SEQ ID NO: 55) | |

The target markers are then detected according to the technique summarized in example 1. This detection of course reveals around ten markers present in each product, but it is by comparing with the coding markers that one knows how to draw the relevant information.

### Example 4

### Labeling a Medicine According to the Method of the Invention

This example shows how to mark medicines which are in the form of caplets such as those employed for numerous medicinal formulas, according to the labeling method of the present invention. The protocol of example 1 is used.

IV.A. Injecting the Polynucleotides into the Capsules

4.a.1 Principle

The markers are first placed in an ethanol solution (80%) then incorporated during the caplet manufacturing process, at a final concentration of 10⁻¹² mole per caplet of 400 mg for the target and decoy markers, and at a concentration of 10⁻¹⁴ moles per caplet for the coding markers. For this example, the caplets do not contain any active principle.

4.a.2 Material
Synthesis polynucleotides (Eurofins MWG GmbH), 100 pmol/μL
Circular single-stranded deoxyribonucleic acid (coding markers)
Nuclease-free, de-ionized H₂O
Beta-Lactose, Sigma Aldrich (registered trademark) #L3750-500G
Corn starch, Sigma Aldrich (registered trademark) #S4180-500G
Calcium dihydrogen phosphate, Sigma Aldrich (registered trademark) #307645
Sugar syrup
Magnesium stearate, Sigma Aldrich (registered trademark) #26454-1KG

4.a.3 Methods

a. Preparation of the Marker Solution

The markers are prepared in the same manner as in example 1.

A preliminary mixture is however achieved in an 80% ethanol solution for the incorporation of the polynucleotides in the caplets.

b. Preparation of Caplets

The caplets are prepared from 300 g of granules. The different powders composing the granules are first of all weighed then mixed (“Lodigge” type barrel type high speed granulator), according to the following formula:
Beta-lactose: 120 g
Corn starch: 60 g
Calcium dihydrogen phosphate: 120 g

The wetting solution is then prepared from 100 g of sugar syrup and 750 μL of the solution of the markers. Then gradually add this wetting solution to the mixture of powders, until obtention of a humid mass having the aspect of coarse-grained semolina, then granulate the previous mixture to obtain a humid vermicular-like granule (using a oscillating granulator).

The granule is then dried at a temperature of 60° C. until a hygrometry from 4 to 6% is obtained, then sieved in a sieve column in order to remove the fine particles. After lubrication using 1% magnesium stearate, the granules are loaded into a press and then compressed thereto.

### IV.B. Extraction and Detection of the Markers

The markers are detected in the same manner as in example 1, except for the extraction step of polynucleotides.

4.b.1 Extraction Principle

The markers are extracted by milling the caplets, then recovering the polynucleotides in aqueous phase.

4.b.2 Material
Labeled caplets.
Heating block or water bath.
H20 demineralized, nuclease-free.

4.b.3 Method

The caplets are milled using mortar and a pestle, such as to obtain a very fine powder. This powder is then mixed with 1 mL of distilled water in a tube of 1.5 mL (Eppendorf, registered trademark). After heating for 15 minutes at 70° C., the tube is centrifuged for 5 minutes at 5000 rcf in order to remove the solid particles. The aqueous phase is then taken up in a new tube for the polynucleotide authentication step. The polynucleotides may then be detected according to the method described in the example 1.

### Example 5

### Labeling of a Food Product According to the Method of the Invention

This example shows how to mark then extract the markers from a food product, such as pizza dough. The markers are injected in the fresh dough and during preparation. They may be detected afterwards in the end product, ready for consumption, cooked or not.

### V.A Labeling of the Dough

5.a.1 Principles

The markers are pre-diluted in demineralized water. Mixtures of target, coding and decoy polynucleotides are used as shown in example 1. Then they are incorporated to this pizza dough recipe, that is then put to cook.

5.a.2 Material
Synthesis polynucleotides (Eurofins MWG GmbH), 100 pmol/μL
Circular single-stranded deoxyribonucleic acid (coding markers)
Nuclease-free de-ionized H₂O
Ingredients for pizza dough: T45 flour ““All-purpose wheat flour for tasty preparations” Francine (registered trademark), olive oil “Extra virgin cold pressed olive oil” Carapelli (registered trademark), yeast “baker's yeast for bread” Francine (registered trademark), sugar “white castor sugar” Daddy (registered trademark), salt “essential white fine iodized salt” Cérébos (registered trademark).

5.a.3 Methods

A preliminary solution of target, coding and decoy markers is added, prepared as summarized in example 1, and added using a Pipette P200 (Gilson Pipetman, registered trademark) to the pizza dough during its manufacture, in sufficient quantity for obtaining a final concentration in markers of 1^{E}-10 moles/kilogram (for each non decoy marker, namely at 2^{E}-9 moles/kilogram of polynucleotides). The following table, (table 5.1) indicates the various quantities of ingredients for obtaining 828 grams of raw pizza dough.

TABLE 5.1
Manufacturing labeled pizza dough

| Ingredient | Quantity or Volume | |
|---|---|---|
| Flour | 500 | g |
| Olive oil | 50 | g |
| Yeast | 12 | g |
| Sugar | 8 | g |
| Salt | 8 | g |
| Water | 250 | mL |
| Preliminary solution of | 166 | μL |
| polynucleotides | | |

The dough is then baked in the oven, during 15 minutes at an average temperature of 240° C.

### V.B Extraction of the Markers

The markers are extracted by dissolution of a portion of cooked dough in demineralized water: 1 gram of cooked dough is reduced to powder, then taken up in 10 mL of water. The whole is then vigorously mixed then placed to heat for 15 minutes at 94° C. After centrifugation for 5 minutes at 10 000 rcf, the aqueous phase is recovered and stored at 4° C. for the detection of the markers. These markers have been detected according to the technique shown in example 1.

### Example 6

### Labeling Tobacco According to the Method of the Present Invention

This example shows how to mark tobacco. It is labeled using polynucleotides such as shown in example 1 by direct absorption of the tobacco.

### VI.A Labeling the Tobacco

6.a.1 Principle

The labeling pertains to tobacco of cigarettes called “blondes” (Virginia tobacco cigarettes). On this sample is absorbed a mixture of target polynucleotides, coding nucleotides and decoy polynucleotides such as described in example 1.

6.a.2 Material
Synthesis polynucleotides (Eurofins MWG GmbH), 100 pmol/μL
Circular single-stranded deoxyribonucleic acid (coding markers)
Ethanol (denatured Ethanol anhydrous, Sigma-Aldrich #676829)
Nuclease-free de-ionized H₂O
Marlboro cigarettes (registered trademark), Philip Morris Products Plc.

6.a.3 Methods

Tobacco is labeled by the polynucleotide solution prepared according to the method described in example 1.

A gram of tobacco cigarette extract is labeled such as to obtain, in the end, 10⁻¹² moles of marker per gram of tobacco: a sufficient quantity of pre-labeling solution is injected using a pipette P200 (Gilson Pipetman, registered trademark) directly on the tobacco, then the whole is vigorously mixed, at a temperature of 37° C. for 15 minutes.

The tobacco, thus labeled is then kept at a temperature of 16° C. and 70% hygrometry.

### VI.B Extraction and Identification of the Markers

The markers are extracted by soaking the labeled tobacco in demineralized water: a gram of tobacco is set to soak in 10 mL of de-ionized distilled water, then put to heat for 15 minutes at 94° C.

After centrifugation for 5 minutes at 10 000 rcf, the aqueous phase is recovered and stored at 4° C. for the detection of the markers. These markers may be detected according to the technique shown in example 1.

### Example 7

### Labeling of a Hydrocarbon According to the Method of the Invention

This example shows how to mark a hydrocarbon (crude oil) and which technique may be used to extract the markers and detect the labeling.

### VII.A Labeling the Hydrocarbon

7.a.1 Principles

In this example, a sample of crude oil is labeled using polynucleotides which were previously prepared in a polar organic solvent. The labeling is composed of target polynucleotides, coding polynucleotides and decoy polynucleotides such as described in example 1.

7.a.2 Material
Synthesis polynucleotides (Eurofins MWG GmbH), 100 pmol/μL
Circular single-stranded deoxyribonucleic acid (coding markers)
Nuclease-free de-ionized H₂O
Dimethylsulfoxide (DMSO), Euromedex ref UD8050-A
crude oil (IFP)

7.a.3 Methods

A solution of single-stranded target polynucleotides, coding and decoy are prepared such as described in example 1.

A second preliminary mixture is then achieved in DMSO before inserting the markers in the oil.

The second preliminary solution of markers is then mixed to the oil, using a precision pipet (Gilson Pipetman, registered trademark) in sufficient quantity for obtaining a final concentration of markers of 10⁻¹⁰ moles/L.

The labeled oil is then stored at ambient temperature, with a view to identifying the markers.

### VII.B Extraction and Detection of the Markers

7.b.1 Principle

The markers are extracted from their apolar environment, then recovered in aqueous environment with the purpose of then using an identification technique.

7.b.2 Material
UltraPure phenol (trademark) Buffer-Saturated Phenol, Invitrogen.
chloroform/amyl alcohol (24:1)
3M Sodium acetate
95% frozen ethanol (Carlo Erba Rectapur), 75%
Nuclease-free de-ionized water
Hexane (Sigma, ref H9379-1L)

7.b.3 Methods

The technique used is that of phenol-chloroform extraction. For the example, the markers are extracted from 500 μL of crude oil.
Add 2 volumes of Hexane.
Add ½ volume of phenol (250 μL), ½ volumes of chloroform (250 μL), and ½ volume of water (250 μL), mix very vigorously for 30 seconds. Centrifuge for 30 minutes at 10 000 rcf.
Transfer the aqueous phase to a new tube, and add 1 volume of chloroform. Mix very vigorously and centrifuge for 20 minutes at 10 000 rcf. Repeat this step 5 times.
Transfer the aqueous phase to a new tube, and add 1/10 volume of 3M sodium acetate and 2.5 volumes of frozen ethanol. Blend then incubate for 1 hour at −20° C.
Centrifuge for 10 minutes at 10 000 rcf. Throw away the supernatant and carefully wash the pellet with 200 μL of nuclease-free deionized water.

These markers may be detected according to the technique shown in example 1.

### Example 8

### Labeling a Fresh Food Product According to the Method of the Invention

In this example, a fresh food product is labeled using a mixture of polynucleotides. The product is a dairy product: a yogurt.

### VIII. A Labeling Yogurt

8.a.1 Principles

The markers are pre-diluted in demineralized water. The labeling is composed of target polynucleotides, coding nucleotides and decoy nucleotides such as described in example 1.

8.a.2 Material
Synthesis polynucleotides (Eurofins MWG GmbH), 100 pmol/μL
Circular single-stranded deoxyribonucleic acid (coding markers)
Nuclease-free deionized H₂O
Activia yogurt (registered trademark) by Danone (registered trademark)

8.a.3 Methods

A preliminary solution of target, coding and decoy polynucleotides is added to the yogurt, in the mass, in sufficient quantity for obtaining a final concentration in markers of 1^{E}-10 moles/kilogram (for each non decoy marker, namely at 2^{E}-9 moles/kilogram of polynucleotides) using a pipette P10 (Gilson Pipetman, registered trademark), 2 μL of preliminary solution is then injected in the mass of the product. The whole is then homogenized well using a sterile spatula.

The labeled yogurt is then stored at 4° C. while waiting to identify the markers.

### VIII.B Extraction of the Markers

The markers are extracted by dissolution of a portion of yogurt in demineralized water: 1 gram of yogurt is taken up in 10 mL of water. The whole is then mixed vigorously then place to heat for 15 minutes at 94° C. After centrifuging for 5 minutes at 10 000 rcf, the aqueous phase is recovered and stored at 4° C. for the detection of the markers. These markers may be detected according to the technique shown in example 1.

### Example 9

### Labeling a Beverage According to the Method of the Invention

A non-alcoholic soft drink is labeled using polynucleotides such as described in example 1: Orangina (registered trademark), Schweppes International Limited.

### IX.A Labeling of the Beverage

9.a.1 Principles

The target, coding and decoy markers are pre-diluted in demineralized water. They are then directly mixed into the drink.

9.a.2 Material
Synthesis polynucleotides (Eurofins MWG GmbH), 100 pmol/μL
Circular single-stranded deoxyribonucleic acid (coding markers)
Nuclease-free, de-ionized H₂O
Orangina (registered trademark), Schweppes International Limited.

9.a.3 Methods

A preliminary mixture is achieved in deionized water before inserting the markers into the drink.

The preliminary solution of markers is mixed to the drink, in sufficient quantity for obtaining a final concentration in markers of 1^{E}-10 moles/L (for each non decoy polynucleotide, namely 2^{E}-9 moles/L of polynucleotides).

The labeled drink is then stored at 4° C. in a hermetic recipient.

### IX.B Extraction of the Markers

The markers are extracted using the phenol-chloroform technique shown in example 1.

These markers may be detected according to the technique exhibited in example 1.

### Example 10

### Labeling a Paper Base According to the Method of the Invention

This example shows how to label then extract markers from a paper base. The markers are directly absorbed by the paper. They are detected afterwards by dissolving the labeled paper.

### X.A Labeling the Paper

10.a.1 Principles

The target, coding and decoy polynucleotides such as described in example 1 are pre-diluted in demineralized water. A drop is thus absorbed on the surface of the sheet of paper.

10.a.2 Material
Synthesis polynucleotides (Eurofins MWG GmbH), 100 pmol/μL
Circular single-stranded deoxyribonucleic acid (coding markers)
Deionized nuclease-free H2O.
Paper disc “1 QUALITATIVE Filter Paper”, Whatman (registered trademark).

10.a.3 Methods

A preliminary mixture such as summarized in example 1 is carried out in de-ionized water, before depositing it on the sheet of paper.

The preliminary solution of markers is thus deposited on the paper, such that 1 E-12 moles of markers are deposited. 1 μL of preliminary solution is deposited using a precision pipette P10 (Gilson Pipetman, registered trademark), thus leading to the formation of a 5 mm-diameter disk.

The paper is the dried in the open air then kept with a view to detect the markers.

### X.B Extraction of the Markers

The markers are extracted by dissolution of a section of paper in demineralized water: 1 cm² of paper containing the disk of markers is cut into very small pieces, then plunged into 10 mL of water. The whole is then put to heat for 15 minutes at 94° C.

The pulp of the paper is mixed very vigorously, then homogenized by suction-discharge using a pipette. After centrifugation during 5 minutes at 2000 rcf, the aqueous phase is recovered and stored at 4° C. for the detection of the markers. These markers may be detected according to the technique shown in example 1.

### Example 11

### Olfactory and Ageing Tests on a Labeled Product According to the Method of the Invention

Stability tests over time and ageing tests have been jointly carried out with a cosmetic industrialist. Olfactory tests have enabled to check that by carrying out labeling according to the present invention, the addition of target, coding and decoy polynucleotides does not modify the physico-chemical and olfactory properties of the perfumes. These tests have been carried out on several perfumes, and according to several conditions: one month at 5° C. which has served as reference, one month at 50° C., which simulated an accelerated ageing of the perfume, as well as a month exposed to the light of day.

After ageing, tests have shown that the solution containing polynucleotides keep the same physico-chemical and olfactory properties as the solutions which do not contain polynucleotides and thus, whatever the ageing method. Furthermore, for each bottle, the markers/markers have been extracted, and then identified successfully.

### Example 12

### Olfactory and Ageing Tests on a Labeled Product According to the Method of the Invention

Accompanying FIG. 8 represents a detection schema of a labeling according to the invention and the authentication of a labeled product according to the invention.

In this figure:

1—The coding markers are identified. The first level of authentication of the product consists in their presence. If the product does not contain coding markers, the product is a counterfeit.

2—The decoy and target markers are then identified in the product. On this fig. the product has been labeled with a series of 20 putative target and decoy polynucleotides. Thus, it contains between 1 and 20 markers selected from this batch. At this step of the method, the target and decoy markers are not differentiated (it is impossible to say whether a polynucleotide is a target polynucleotide or a decoy polynucleotide).

3—The nature of the coding polynucleotide detected during the first step (here, polynucleotide A) is then sent to the labeling provider. Thanks to this information, the labeling provider recuperates the decipher key of the labeling from a database, thus allowing him/her to differentiate between the decoy polynucleotides and the target ones from the series of decoy and target polynucleotides.

4—Using the decipher key, the user is only concerned with the target polynucleotides. He/she may thus, read the code composed by the presence or absence of the target polynucleotide(s) (in this example, the code is −,−,+ and −).

5—The reading of the code thus enables the user to check, using a secured database, whether the identification code does indeed correspond to the product of interest. In the opposite case, it may consist in an illegal reproduction, of a mixture or a misappropriation of the product.

### REFERENCE LIST

[1] Molecular Cloning, Maniatis, Cold Spring-Harbor, 2nd edition, pp C3 to C14.
[2] Smith T F, Waterman M S (1981). Identification of common molecular subsequences, J Mol Biol 1981 Mar. 25; 147(1): 195-7.
[3] Xin W, Zhang Y M, Xiao J H, Huang D W (2003). Construction of linear functional expression elements with DNA fragments created by site-specific DNA nickase, N.BpulO I, and exonuclease III, Biotechnol Lett. 2003 November; 25(22): 1913-6.
[4] Caruthers M H, Beaucage S L, Efcavitch J W, Fisher E F, Matteucci M D, Stabinsky Y., (1980) New chemical methods for synthesizing polynucleotides, Nucleic Acids Symp Ser. 1980; (7):215-23.
[5] Wheeler, C. J., L Sukhu, G. Yang, Y. Tsai, C. Bustamente, P. Feigner, J. Norman, M. Manthorpe. (1996). Converting an alcohol to an amine in a cationic lipid dramatically alters the co-lipid requirement, cellular transfection activity and the ultrastructure of DNA-cytofectin complexes. Bioch. Biophys. Acta 1280:1.
[6] Feigner, J. H., R. Kumar, C. N. Sridhar, C. J. Wheeler, Y. S. Tsai, R. Border, P. Ramsey, M. Martin, P. L. Feigner. (1994). Enhanced gene delivery and mechanism studies with a novel series of cationic lipid formulations. J. Biol. Chem. 269:2550
[7] Ogris M, Steinlein P, Carotta S, Brunner S, Wagner E (2001) NA/polyethylenimine transfection particles: influence of ligands, polymer size, and PEGylation on internalization and gene expression, AAPS PharmSci. 2001; 3(3):E21.
[8] Molecular Cloning. Maniatis, Cold Spring-Harbor, 2nd edition, pp E3 à E4.
[9] Molecular Cloning, Maniatis, Cold Spring-Harbor, 2nd edition pp 9.47 à 9.57.
[10] Kabilov M R, Pyshnyi D V, Dymshits G M, Gashnikova N M, Pokrovskii A G, Zarytova V F, Ivanova E M (2002). A new approach to detect a particular DNA sequence by UV-immobilization of its hybridization complex with a highly specific probe resulting from ligation of a tandem of short oligonucleotides in solution, Mol Biol (Mosk). 2002 May-June; 36(3):424-31.
[11] Ramsay G (1998). DNA chips: state-of-the art. Nat Biotechnol. 1998 January; 16(1):40-4.
[12] Ivanovskaia M G, Kozlov I A, Lebedeva I V, Shabarova Z A (1994). A new method of covalent immobilization of oligodeoxyribonucleotides on nylon membranes for hybridization with nucleic acids, Mol Biol (Mosk). 1994 September-October; 28(5): 1176-82.

1. A method for labeling a product, said method comprising a step of adding on or in said product a plurality of single-stranded polynucleotides, said plurality of polynucleotides comprising:
at least one target polynucleotide constituted of a single-stranded polynucleotide of predetermined length and sequence, and
decoy polynucleotides which have identical or different predetermined lengths and identical or different predetermined sequences, said decoy polynucleotides having a length or lengths identical to or different from and sequences different from the sequence of said, at least one, target polynucleotide,
wherein each of the target and decoy polynucleotides does not hybridize with any of the other polynucleotides of said plurality of polynucleotides, and
wherein the polynucleotides of the plurality of polynucleotides are deoxyribonucleic or ribonucleic acid sequences, respectively comprising the same proportion of the four, natural or modified, bases A, C, G and T, or A, C, G and U.

2. The method according to claim 1, wherein said plurality of polynucleotides further comprises at least one recognition polynucleotide constituted of a single-stranded polynucleotide of predetermined length and sequence for identifying the nature and sequence of the, at least one, target polynucleotide, wherein each recognition polynucleotide does not hybridize with any of the other polynucleotides of said plurality of polynucleotides.

3. The labeling method according to claim 1, wherein said polynucleotides of the plurality of polynucleotides are circular or linear.

4. The labeling method according to claim 1, wherein at least two target polynucleotides are used, one being a circular polynucleotide and the other a linear polynucleotide

5. The labeling method according to claim 1, wherein the linear polynucleotides comprise a variable end from one polynucleotide to the other and a constant end from one polynucleotide to the other.

6. The labeling method according to claim 1, wherein the, at least one, target polynucleotide of the plurality of polynucleotides has a length of 5 to 50 nucleotides.

7. The labeling method according to claim 1, wherein said polynucleotides of the plurality of polynucleotides has a length of 5 to 5000 nucleotides.

8. The labeling method according to claim 1, wherein the step of addition is carried out by adding said plurality of polynucleotides in said product during its manufacture either in or on the end product.

9. The labeling method according to claim 1, wherein the step of addition is carried out by adding said plurality of polynucleotides at the surface of said product.

10. The labeling method according to claim 1, wherein an encapsulation step of said plurality of polynucleotides in lipid vectors is carried out prior to the addition step.

11. The labeling method according to claim 1, wherein the introduction of said plurality of polynucleotides is carried out on or in a component of said product.

12. The labeling method according to claim 1, wherein the concentration of the plurality of polynucleotides after its addition in said product is of 10⁻⁶ moles to 10⁻¹⁸ moles/dm3.

13. A labeled product obtainable by a method according to claim 1.

14. The labeled product according to claim 13, said labeled product is perfumes, cosmetics, hygiene products, food products, flavorings, plant extracts, tobacco, beverages, textiles, leathers, medicines, powders, varnishes, inks, food products, hydrocarbons, papers, paints, or chemical products and compounds.

15. A method for detecting the labeling of a product obtainable by a method according to claim 1, said method comprising an analyzing step of the plurality of polynucleotides enabling to detect specifically the, at least one, target polynucleotide, comprising the following successive steps of:
(i) placing in contact the plurality of polynucleotides with a solid support whereon probe sequences are fixed, these probe sequences being complementary to one of the said ends of said, at least one, target polynucleotide of the plurality of polynucleotides of the labeling of the product, the placing in contact allowing the target polynucleotides to be fixed on the support by hybridization with the complementary probe sequences fixed on the support;
(ii) eliminating the polynucleotides non hybridized by step (i);
(iii) detecting the presence of the target polynucleotides on the support; and
(iv) comparing the results of step (iii) with the contents of a database enabling to identify and authenticate said product.

16. The method according to claim 15, further comprising the following steps, before the analyzing step:
(a) taking a sample of the product; and
(b) extracting the plurality of polynucleotides from said sample, the analyzing step being achieved on the plurality of polynucleotides extracted from said sample.

17. The detection method according to claim 15 wherein the analyzing step comprises a polymerization chain reaction of the target polynucleotides.

18. The detection method according to claim 15, wherein the analyzing step is carried out by immunodetection.

19. The method for detecting the labeling according to claim 15, wherein the polynucleotides complementary to the target polynucleotides are fixed to the solid support by means of a biotin/streptavidin connection.

20. The method for detecting the labeling according to claim 15, wherein the polynucleotides complementary to the target polynucleotides are fixed to the support by forming a covalent bond to a non-charged nylon membrane, said membrane forming the solid support.

21. The method for detecting the labeling according to claim 15, wherein the target polynucleotides fixed to the support are detected by means of polynucleotides labeled by a labeling agent and complementary to the other end of the target polynucleotides, the labeling agent is a fluorochrome, a colloidal gold particle or an enzyme.

22. The method for detecting the labeling according to claim 15, wherein the database enables to determined the origin of said product.

23. The method for detecting the labeling according to claim 15, wherein the database enables to identify a counterfeit of the original product.

24. The method for detecting the labeling according to claim 15, wherein the extraction such as defined in step (b) is a phenol-chloroform extraction.

25. The detection method according to claim 15, wherein analyzing step comprises a retro-transcription of ribonucleic acid into deoxyribonucleic acid when the target polynucleotides are ribonucleic acid.

26. The detection method according to claim 15, wherein the analyzing step comprises a step of sequencing the target polynucleotides.
